(19) United States
(12) Patent Application Publication
(10) Pub. No.: US 2025/0205050 A1
(43) Pub. Date: Jun. 26, 2025

(54) PROSTHETIC VALVE, CATHETER APPARATUS, AND RELATED METHODS

(71) Applicant: THUBRIKAR AORTIC VALVE, INC., Norristown, PA (US)

(72) Inventors: Evan ROUSH, Blue Bell, PA (US); Mark EBERHARDT, Elverson, PA (US); Ali RECBER, Quakertown, PA (US); Mano THUBRIKAR, Schwenksville, PA (US)

(73) Assignee: THUBRIKAR AORTIC VALVE, INC., Norristown, PA (US)

(21) Appl. No.: 18/848,443

(22) PCT Filed: Mar. 23, 2023
(86) PCT No.: PCT/US2023/016031

§ 371 Date: Sep. 18, 2024

Related U.S. Application Data
(60) Provisional application No. 63/322,708, filed on Mar. 23, 2022.

(51) Int. Cl. A61F 2/24

(52) U.S. Cl. CPC A61F 2/2436

(57) ABSTRACT

A catheter apparatus and a method for using the catheter apparatus to implant a prosthetic aortic valve into a patient. The catheter apparatus includes a handle assembly and a catheter assembly. The catheter assembly has an outer sheath which defines a sheath cavity within which a pusher is positioned, an outer shaft coupled to a slider of the handle assembly and to the sheath cavity, and an inner shaft to which the pusher is attached. A self-expanding prosthetic valve is positioned in the outer sheath and coupled to the pusher. Actuation of the handle causes the slider and the outer sheath to move in a proximal axial direction to expose and deploy the self-expanding prosthetic valve. The self-expanding prosthetic valve may have tabs that are engaged with indents of the pusher such that the tabs are released sequentially during deployment.

### CROSS-REFERENCE TO RELATED APPLICATION

The present application claims the benefit of priority to U.S. Provisional Patent Application Ser. No. 63/322,708, filed Mar. 23, 2022, the entirety of which is incorporated herein by reference.

### BACKGROUND

Heart valve disease continues to be a significant cause of morbidity and mortality. Heart valve replacement has become a routine surgical procedure for patients suffering from valve regurgitation or stenotic calcification of the leaflets. Until recently, the vast majority of heart valve replacements entailed a full sternotomy and placing the patient on cardiopulmonary bypass. Traditional open surgery inflicts significant patient trauma and discomfort, requires extensive recuperation times, and may result in life-threatening complications. To address these concerns, within the last fifteen years efforts have been made to perform cardiac valve replacements using minimally-invasive techniques, such as a percutaneous entry with a transluminal delivery. These surgical techniques, generally referred to as Transcatheter Aortic Valve Implantations (TAVI) or Transcatheter Aortic Valve Replacements (TAVR), use a catheter to deliver a prosthetic valve to an implantation site using a patient's lumen of the vascular system. There remains a need for improvements to the prosthetic valves used in TAVI/TAVR and the mechanisms used to deliver them into the patient's body.

### SUMMARY

The present invention is directed to a catheter apparatus, a self-expanding prosthetic valve for use with the catheter apparatus, a method of loading the catheter apparatus with the self-expanding prosthetic valve, and a method for implanting the self-expanding prosthetic aortic valve into a patient using the catheter apparatus.

In one aspect, the invention may be a catheter apparatus comprising: a handle assembly comprising: a handle housing comprising a longitudinal axis, an outer surface and an inner surface that defines a handle cavity; a rotating member positioned around a portion of the outer surface of the handle housing and configured to rotate relative to the handle housing, the rotating member comprising a threaded inner surface; a slider at least partially positioned within the handle cavity and comprising a threaded portion that mates with the threaded inner surface of the rotating member; an inlet port defining a passageway into the handle cavity; and a flushing tube having a coiled configuration, the flushing tube comprising a first end fluidly coupled to the inlet port, a second end fluidly coupled to the slider, and a lumen extending from the first end to the second end; and wherein rotation of the rotating member causes the slider to move axially within the handle cavity, and wherein the flushing tube compresses as the slider moves in a proximal axial direction and expands as the slider moves in a distal axial direction.

In another aspect, the invention may be a catheter apparatus comprising: a catheter assembly comprising: an inner shaft comprising a lumen; a first axial wire and a second axial wire disposed within the lumen of the inner shaft, wherein when the inner shaft is bent into a U-shaped bend configuration the first and second axial wires are aligned on a vertical plane; and a pusher comprising a plurality of indentations that are configured to receive tabs of a frame of a prosthetic aortic valve, wherein the pusher is coupled to the inner shaft so that one of the plurality of indentations is aligned on the vertical plane when the inner shaft is bent into the U-shaped bend configuration.

In yet another aspect, the invention may be a catheter apparatus comprising: a catheter assembly comprising: an inner shaft comprising a lumen; a first axial wire and a second axial wire disposed within the lumen of the inner shaft, wherein when the inner shaft is bent into a U-shaped bend configuration the first and second axial wires are aligned on a vertical plane; and a pusher comprising a plurality of indentations that are configured to receive tabs of a frame of a prosthetic aortic valve, wherein the pusher is coupled to the inner shaft so that one of the plurality of indentations is angularly offset 90° from the vertical plane.

In a further aspect, the invention may be a catheter apparatus comprising: a handle assembly; and a catheter assembly coupled to the handle assembly, the catheter assembly comprising a pusher configured for engagement with a prosthetic aortic valve to facilitate implantation of the prosthetic aortic valve into a patient, the pusher comprising at least one indentation that is configured to receive a tab of a frame of the prosthetic aortic valve, the at least one indentation comprising a proximal end surface that is sloped.

In a still further aspect, the invention may be a catheter assembly comprising a pusher configured for engagement with a prosthetic aortic valve to facilitate implantation of the prosthetic aortic valve into a patient, the pusher comprising at least one indentation that is configured to receive a tab of a frame of the prosthetic aortic valve, the at least one indentation comprising a proximal end surface that is sloped.

In another aspect, the invention may be a catheter system comprising: a catheter apparatus comprising: a catheter assembly comprising: a deployment assembly comprising an outer sheath defining a sheath cavity, a distal tip assembly configured to close an open distal end of the outer sheath, and a pusher that is located within the sheath cavity when the outer sheath is in a non-deployed position, the pusher and the distal tip assembly spaced apart from one another by an axial space, the pusher comprising a plurality of indentations; a handle assembly operably coupled to the catheter assembly and configured to move the outer sheath in a proximal axial direction to alter the outer sheath from the non-deployed position into a deployed position; a self-expanding prosthetic valve in a radially compressed state located within the axial space between the pusher and the distal tip assembly, the self-expanding prosthetic valve comprising a frame and a valve component coupled to the frame, the frame comprising a tubular body portion and a plurality of tabs extending from an end of the tubular body portion, each of the tabs nesting within one of the plurality of indentations of the pusher; and wherein actuation of the handle assembly causes the outer sheath to move in the proximal axial direction to alter the outer sheath from the non-deployed position to the deployed position, and wherein when the plurality of tabs remain in the sheath cavity and the tubular body portion is removed from the sheath cavity, no more than 70% of the self-expanding prosthetic valve is in a fully expanded state.

In still another aspect, the invention may be a method for implanting a self-expanding prosthetic aortic valve in a patient, the method comprising: loading a self-expanding prosthetic aortic valve into a catheter apparatus with the self-expanding prosthetic aortic valve in a radially compressed state; inserting a distal end of the catheter apparatus into a vasculature of a patient until the self-expanding prosthetic aortic valve is positioned at an implantation location, the catheter apparatus comprising an outer sheath having a sheath cavity within which the self-expanding prosthetic aortic valve and a pusher are located, the self-expanding prosthetic aortic valve comprising a tubular body portion and a plurality of tabs of identical length extending from an end of the tubular body portion, the plurality of tabs being in engagement with the pusher; moving the outer sheath in a proximal axial direction while the self-expanding prosthetic aortic valve and the pusher are in a fixed axial position; and wherein the plurality of tabs are released from the pusher in a sequential manner as the plurality of tabs become removed from the sheath cavity to deploy the self-expanding prosthetic aortic valve into the patient during the movement of the outer sheath in the proximal axial direction.

In a further aspect, the invention may be a method of loading a self-expanding prosthetic aortic valve into a catheter apparatus, the method comprising: radially compressing a self-expanding prosthetic aortic valve with a radial compression device; positioning the radially compressed self-expanding prosthetic aortic valve onto a pusher of a catheter assembly of the catheter apparatus so that the self-expanding prosthetic aortic valve is engaged with the pusher; sliding the pusher of the catheter apparatus in a proximal axial direction until the pusher and the radially compressed self-expanding prosthetic aortic valve are disposed within a sheath cavity of an outer sheath of the catheter apparatus, wherein a proximal cap assembly of the catheter apparatus slides in the proximal axial direction during the sliding of the pusher in the proximal axial direction; rotating a rotating member of a handle assembly of the catheter apparatus in a first rotational direction to move the proximal cap assembly in a distal axial direction until the proximal cap assembly is adjacent to a proximal end of a handle housing of the handle assembly; and coupling the proximal cap assembly to the handle housing to prevent further axial movement of the proximal cap assembly to axially fix a position of the pusher and the self-expanding prosthetic aortic valve.

In a still further aspect, the invention may be a self-expanding prosthetic valve for implantation into a body lumen, the self-expanding prosthetic valve comprising: a frame comprising: a tubular body portion having a proximal end, a distal end, and a first height measured between the proximal and distal ends; and a plurality of tabs extending from the proximal end of the tubular body portion, each of the plurality of tabs having a second height measured from the proximal end of the tubular body portion to a terminal end of the tab; a valve component coupled to the frame; and wherein a ratio of the first height to the second height is between 5.2:1 and 6.6:1.

In still another aspect, the invention may be a catheter system comprising: a catheter apparatus comprising: a handle assembly comprising: a handle housing having an outer surface and an inner surface defining a handle cavity; a rotating member configured to rotate around the handle housing, the rotating member comprising a threaded inner surface; and a slider at least partially positioned within the handle cavity and comprising a threaded portion that mates with the threaded inner surface of the rotating member; a catheter assembly comprising: a deployment assembly comprising a distal tip assembly, an outer sheath defining a sheath cavity, and a pusher that is located within the sheath cavity of the outer sheath when the outer sheath is in a non-deployed position, the pusher comprising a plurality of indentations; an outer shaft having a proximal end that is coupled to the slider of the handle assembly and a distal end that is coupled to the outer sheath; and an inner shaft at least partially located within a lumen of the outer shaft, the inner shaft having a proximal end that is coupled to the handle housing and a distal end that is coupled to the distal tip assembly, the inner shaft being in a fixed axial position relative to the handle assembly; a self-expanding prosthetic valve in a radially compressed state located within the sheath cavity between the pusher and the distal tip assembly, the self-expanding prosthetic valve comprising a tubular body and a plurality of tabs protruding from an end of the tubular body, each of the plurality of tabs having an identical length, and each of the plurality of tabs nesting within one of the indentations of the pusher; and wherein rotation of the rotating member of the handle assembly relative to the handle housing in a first rotational direction causes the slider, the outer shaft, and the outer sheath to move in a proximal axial direction to expose the self-expanding prosthetic valve which radially expands upon being fully exposed, and wherein the plurality of tabs of the self-expanding prosthetic valve are released from the pusher in a sequential manner.

In yet another aspect, the invention may be a method for implanting a self-expanding prosthetic aortic valve in a patient, the method comprising: loading a self-expanding prosthetic aortic valve into a catheter apparatus with the self-expanding prosthetic aortic valve in a radially compressed state, the catheter apparatus comprising a deployment assembly that comprises an outer sheath and a pusher, the outer sheath having a sheath cavity within which the self-expanding prosthetic aortic valve and the pusher are located, the self-expanding prosthetic aortic valve comprising a tubular body portion and a plurality of tabs extending from an end of the tubular body portion, the plurality of tabs being in engagement with the pusher; inserting the deployment assembly of the catheter apparatus into the patient until the self-expanding prosthetic aortic valve is positioned at an implantation location, wherein a shaft assembly of the catheter apparatus bends into a U-shape having a first leg and a second leg whereby a first circumferential portion of an outer surface of the second leg faces the first leg; moving the outer sheath in a proximal axial direction while the self-expanding prosthetic aortic valve and the pusher are in a fixed axial position to remove the self-expanding prosthetic aortic valve from the sheath cavity and deploy the self-expanding prosthetic aortic valve into the patient at the implantation location; and wherein during deployment of the self-expanding prosthetic aortic valve, one of the plurality of tabs is circumferentially located along the first circumferential portion of the outer surface of the second leg of the shaft assembly of the catheter apparatus.

In another aspect, the invention may be a catheter apparatus comprising: a catheter assembly comprising: an inner shaft comprising a lumen; a first axial wire and a second axial wire disposed within the lumen of the inner shaft, wherein when the inner shaft is bent into a U-shaped bend configuration the first and second axial wires are aligned on a vertical plane; and a pusher comprising a plurality of indentations that are configured to receive tabs of a frame of a prosthetic aortic valve, wherein the pusher is coupled to the inner shaft so that one of the plurality of indentations is angularly offset between 80° and 100° from the vertical plane.

In yet another aspect, the invention may be a catheter apparatus comprising: a catheter assembly comprising: an inner shaft comprising a lumen; a wire assembly disposed within the lumen of the inner shaft to control a bend configuration of the inner shaft such that when the inner shaft is bent into a U-shaped bend configuration, first and second legs of the inner shaft are aligned on a vertical plane; and a pusher comprising a plurality of indentations that are configured to receive tabs of a frame of a prosthetic aortic valve, wherein the pusher is coupled to the inner shaft so that one of the plurality of indentations is angularly offset between 80° and 100° from the vertical plane.

In another aspect, the invention may be a catheter apparatus comprising: a catheter assembly comprising: an inner shaft comprising a lumen; an axial wire assembly disposed within the lumen of the inner shaft, the axial wire assembly comprising a first wire portion and a second wire portion that are aligned on a vertical plane when the inner shaft is bent into a U-shaped bend configuration; and a pusher comprising a plurality of indentations that are configured to receive tabs of a frame of a prosthetic aortic valve, wherein the pusher is coupled to the inner shaft so that one of the plurality of indentations is angularly offset between 80° and 100° from the vertical plane.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a frame of a prosthetic aortic valve in accordance with an embodiment of the present invention;

FIG. 2 is a front view of the frame of the prosthetic aortic valve of FIG. 1;

FIG. 3 is a perspective view of a prosthetic aortic valve which includes the frame of FIG. 1 and a valve portion;

FIG. 4 is a perspective view of a catheter apparatus which includes a handle assembly and a catheter assembly;

FIG. 5 is a cross-sectional view taken along line V-V of FIG. 4;

FIG. 6 is an exploded view of the handle assembly of the catheter apparatus of FIG. 4;

FIG. 6A is a cross-sectional view of a proximal portion of the handle assembly of the catheter apparatus of FIG. 4;

FIG. 7 is an enlarged view of area VII of FIG. 5 with a slider of the handle assembly in a distal position;

FIG. 8 is the enlarged view of FIG. 7 with the slider of the handle assembly in a proximal position;

FIG. 9 is an enlarged view of area IX of FIG. 5 illustrating a deployment assembly of the catheter assembly;

FIG. 10 is an exploded view of a deployment assembly of the catheter assembly of FIG. 4;

FIG. 11 is a perspective view of a pusher of the deployment assembly of FIG. 10;

FIG. 12 is a front view of the pusher of FIG. 11;

FIG. 13 is a cross-sectional view taken along line XIII-XIII of FIG. 12;

FIG. 14 is an illustration of an inner shaft of the catheter assembly of FIG. 4 with the pusher affixed thereto in accordance with one embodiment of the present invention;

FIG. 14A is an illustration of the inner shaft of the catheter assembly of FIG. 4 with the pusher affixed thereto in accordance with another embodiment of the present invention;

FIG. 15 is a cross-sectional view taken along line XV-XV of FIG. 14;

FIG. 16 is a cross-sectional view taken along line XVI-XVI of FIG. 14;

FIG. 16A is a cross-sectional view taken along line XVIA-XVIA of FIG. 14A;

FIG. 16B is a top plan view of a portion of the catheter apparatus, with the pusher in the foreground;

FIG. 16C is a close-up view of the pusher and axial wire from the view of FIG. 16B;

FIG. 17 is a front view of the catheter apparatus of FIG. 4 illustrating removal of a nose cone;

FIG. 18 is the front view of the catheter apparatus of FIG. 17 illustrating rotation of a rotating member of the handle assembly in a first rotational direction to cause the outer sheath to move in a proximal axial direction;

FIG. 19 is the front view of the catheter apparatus of FIG. 18 illustrating decoupling a proximal cap assembly from a remainder of the handle assembly;

FIG. 20 is a perspective view of the catheter apparatus of FIG. 19 illustrating a self-expanding prosthetic aortic valve being held in a radially compressed state and coupled to the catheter apparatus;

FIG. 21 is a partial front view of the catheter apparatus of FIG. 20 illustrating the self-expanding prosthetic aortic valve being slid proximally towards and into the outer sheath while being maintained in the radially compressed state;

FIG. 22 is a front view of the catheter apparatus of FIG. 21 illustrating rotation of the rotating member of the handle assembly in a second rotational direction to cause the deployment assembly and the proximal cap to move in a distal axial direction;

FIG. 23 is a front view of the catheter apparatus of FIG. 22 illustrating the proximal cap being reattached to the remainder of the handle apparatus;

FIG. 24 is a front view of the catheter apparatus of FIG. 23 illustrating the nose cone being reattached to the remainder of the catheter assembly;

FIG. 25 is a view illustrating insertion of the deployment assembly of the catheter apparatus into a vasculature of a patient;

FIG. 26 is a view illustrating the deployment assembly of the catheter apparatus located within the aortic arch of the patient for installation of the prosthetic aortic valve;

FIG. 27 is a cross-sectional view taken along line XXVII-XXVII of FIG. 26 illustrating positioning of tabs of the frame portion of the prosthetic aortic valve relative to inner and outer walls of the aortic arch of the patient;

FIG. 28 is a cross-sectional view taken along line XXVII-XXVII of FIG. 26 illustrating positioning of tabs of the frame portion of the prosthetic aortic valve relative to inner and outer walls of the aortic arch of the patient in accordance with an alternative embodiment; and

FIGS. 29-33 are sequential perspective views of the catheter apparatus illustrating the deployment of the prosthetic aortic valve from the catheter apparatus.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings. which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

As used herein, the words, “proximal” and “distal,” refer to positions or directions closer to and further from, respectively, a physician implanting the replacement aortic valve using the catheter apparatus described herein.

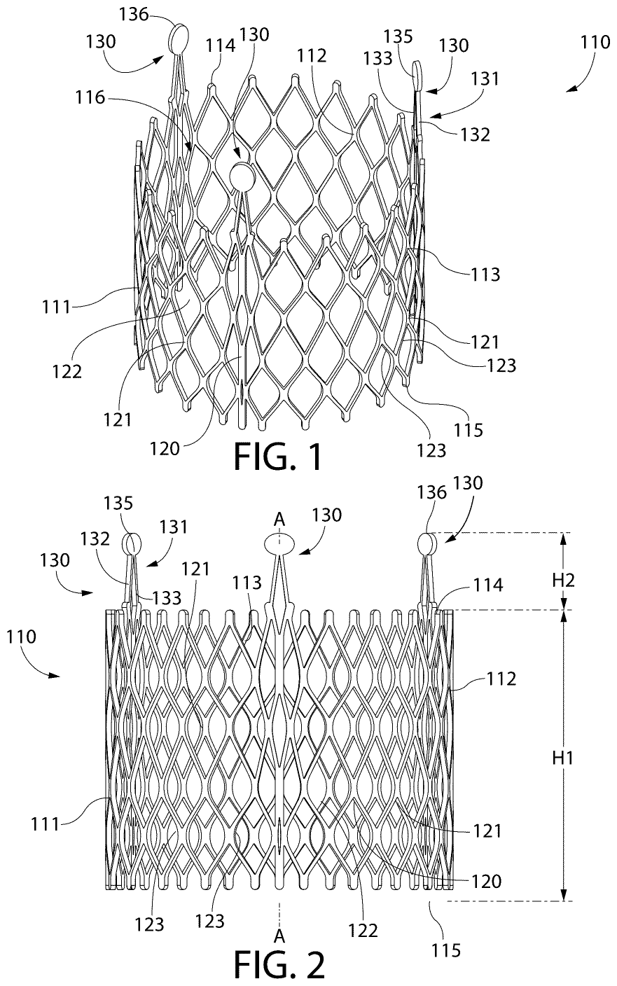

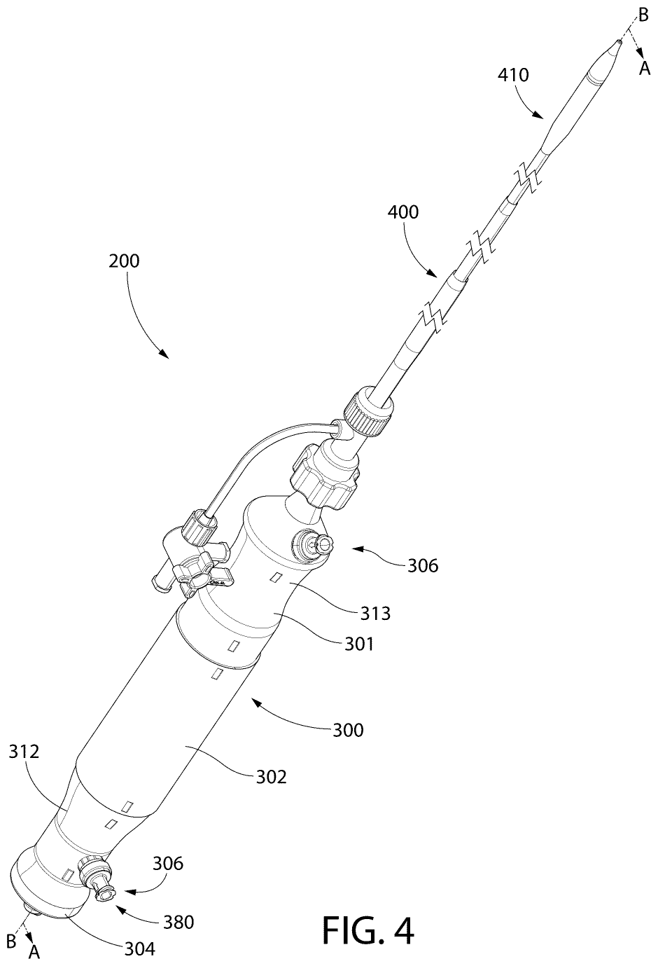

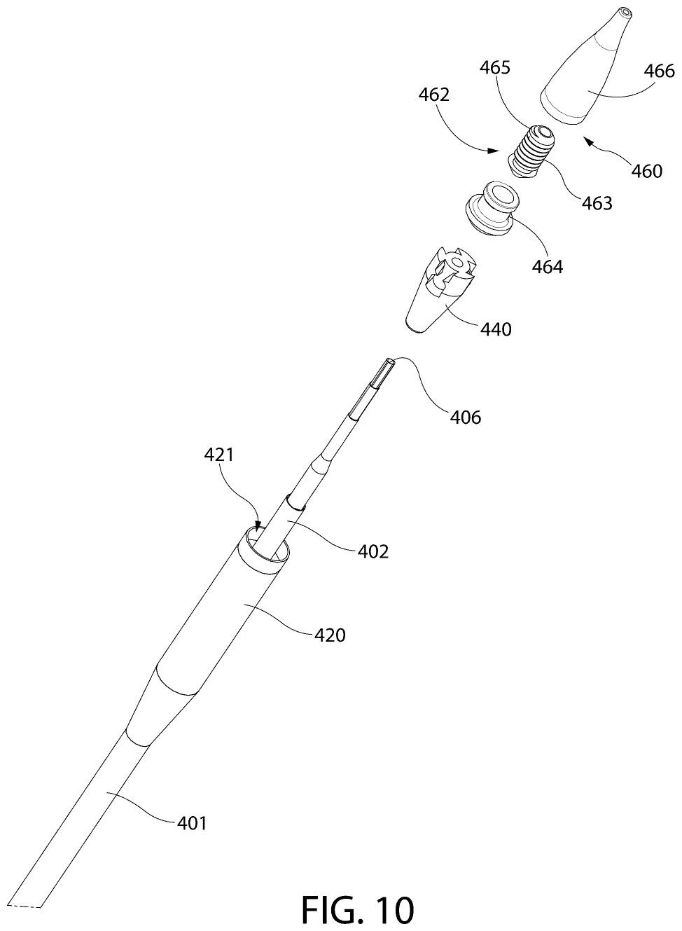

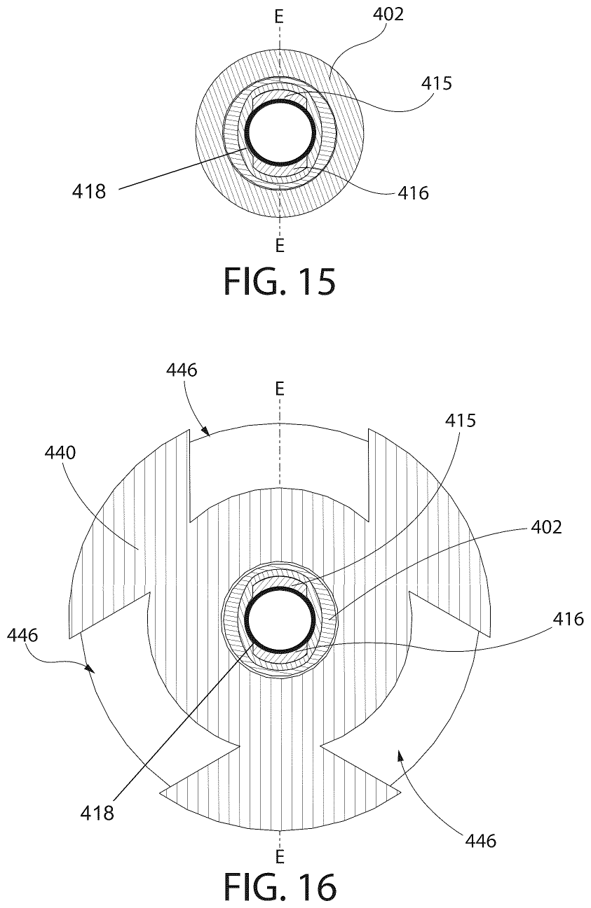

Referring first to FIGS. 1-3, a self-expanding prosthetic aortic valve (hereinafter “the prosthetic valve”) 100 will be described in accordance with an embodiment of the present invention. In the exemplified embodiment, the prosthetic valve 100 is configured to be used with a catheter apparatus 200 as shown in FIGS. 4-24 and described below. However, in other embodiments the prosthetic valve 100 may be able to be deployed and/or otherwise installed in a patient using other catheters or other delivery or installation devices or using processes and methods other than those described herein. That is, in some embodiments the prosthetic valve 100 forms an invention on its own without regard to any specific details about a catheter apparatus or delivery mechanism.

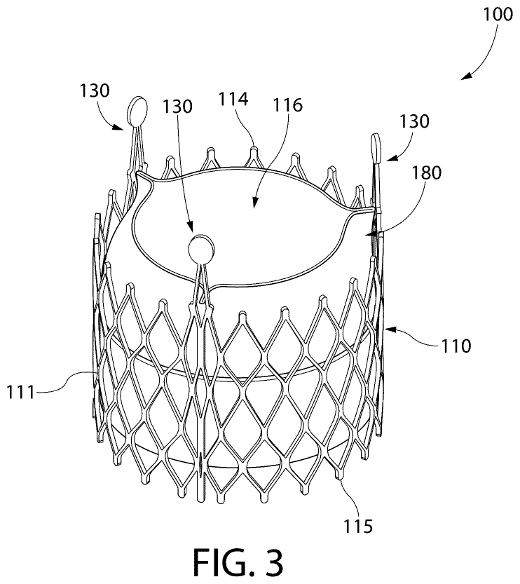

The prosthetic valve 100 generally comprises a frame portion 110 and a valve portion 180. The frame portion 110 is illustrated by itself in FIGS. 1 and 2, and the frame portion 110 and the valve portion 180 are depicted together in FIG. 3. The prosthetic valve 100 is very similar to the prosthetic valve shown and described in U.S. Pat. No. 8,992,599, issued Mar. 31, 2015 to the same assignee as the present application, the entirety of which is incorporated herein by reference. Thus, reference may be made to United States Patent No. 8,992,599 for additional details about the prosthetic valve 100 beyond that which is provided herein. The main difference between the prosthetic valve 100 described herein and the prosthetic valve described in U.S. Pat. No. 8,992,559 is the addition of tabs on the frame of the prosthetic valve 100. For the basic structure of the prosthetic valve, with the exception of the tabs, the disclosure in U.S. Pat. No. 8,992,599 is applicable in accordance with embodiments disclosed herein.

The prosthetic valve 100 is illustrated in its natural state in FIGS. 1-3. That is, in FIGS. 1-3 no forces are acting upon the prosthetic valve 100 to compress or expand the prosthetic valve 100 and it is in its natural shape and size. However, the prosthetic valve 100 is configured to be radially compressed and is intended to be in a radially compressed state when inserted into the catheter apparatus 200 (see, for example. FIG. 20). Upon the prosthetic valve 100 being deployed or released from the catheter apparatus 200, the prosthetic valve 100 automatically radially expands back to its natural or biased state as shown in FIGS. 1 and 2. Thus, the prosthetic valve 100 is a self-expanding valve which can be radially compressed and will automatically radially expand when no longer held under compression. In the exemplified embodiment, both the frame portion 110 and the valve portion 180 are capable of being maintained in at least two configurations, including a first radially compressed configuration (prior to and during delivery) and a second radially expanded (or natural) configuration (after implantation in a patient). The valve portion 180 remains coupled to and disposed within the frame portion 110 both before and after installation in the patient.

The frame portion 110 forms a rigid structure so that the valve portion 180 may be anchored thereto and is capable of maintaining the desired configuration. The frame portion 110 provides the mechanism by which the prosthetic valve 100 may be retained in the prior position and orientation at the desired implantation site. The prosthetic valve 100 may be retained in the proper position and orientation at the desired implantation site by any known means known in the art. none of which are to be considered limiting of the present invention unless specifically recited in the claims. For example, the frame 100 may be anchored directly to the inner wall of the body lumen (or to a secondary frame or stent in which the prosthetic valve 100 is positioned). Such anchoring can be achieved, for example, via known techniques, including without limitation, suturing, stapling, puncturing, clamping or combinations thereof. In the exemplified embodiment, the frame 110 is a self-retaining structure that utilizes its tendency to diametrically expand to a diameter greater than the diameter of the body lumen at the implantation site, thereby creating a compression fit between the prosthetic valve 100 and the body lumen to retain the prosthetic valve 100 in place at the implantation site. The tendency of the frame 110 to diametrically expand can be achieved by forming the frame 110 out of a shape memory material. In some embodiments, the frame 110 is formed of nickel titanium. Other shape memory materials can be utilized in other self-retaining embodiments. In embodiments wherein the frame 110 is not a self-retaining structure, the frame can be constructed of any biocompatible material that is sufficiently rigid to provide the required support to the valve portion 180. Suitable alternate materials include, without limitation, polymers, platinum, stainless steel, chonichrom, or combinations thereof.

The frame portion 110 comprises a tubular body portion 111 having an inner surface 112, an outer surface 113, a proximal end 114, and a distal end 115. The tubular body portion 110 comprises a central axis A-A which extends between the proximal and distal ends 114, 115. The inner surface 112 of the frame portion 110 defines a fluid passageway 116 through which a bodily fluid can flow when the prosthetic valve 100 is installed in a patient. The valve portion 180 is at least partially located within the fluid passageway 116 to control the flow of the bodily fluid through the fluid passageway 116 when the prosthetic valve 100 is installed in the patient. The valve portion 180 is the working component of the prosthetic valve 100 and is alterable between: (1) an open state in which the fluid passageway 116 is open and allows a body fluid to pass therethrough; and (2) a sealed state in which the fluid passageway 116 is sealed and prevents backflow of bodily fluid that has exited the fluid outlet. The valve portion 180 is disposed within and anchored to the tubular body portion 111 of the frame 110 so as to be capable of repetitively alternating between the open and closed states when the prosthetic valve 100 is anchored at the implantation site. The valve portion 180 may comprise three flaps as depicted, or other numbers of flaps as may be appropriate. Additional details about the valve portion 180 may be found in U.S. Pat. No. 8,992,599, which was incorporated herein by reference above.

The tubular body portion 111 of the frame portion 110 may comprise a plurality of posts 120 and a plurality of lattice structures 121 circumferentially extending between and connected to the posts 120. The posts 120 extend from the proximal end 114 to the distal end 115 of the tubular body portion 111 and, in the exemplified embodiment are substantially linear structures that are substantially parallel to the axis A-A. The posts 120 are arranged about the circumference of the tubular body portion 111 in a spaced-apart manner. More specifically, the posts 120 are arranged in an equi-spaced manner about the circumference of the tubular body portion 111. In certain embodiments, the number of posts 120 will correspond with the number of commissures present on the valve portion 180 because the posts 120 provide structures within the tubular body portion 111 to which the commissures are mounted. The tubular body portion 111 may comprise one of the posts 120 for each commissure of the valve portion 180.

In the exemplified embodiment, there are three posts 120 because the valve portion 180 is a tricuspid type valve, thereby having three commissures. However, in alternate embodiments the tubular body portion 111 can include more or less than three posts 120 as desired. Moreover, in certain embodiments, it is possible that the number of posts 120 can be greater than the number of commissures of the valve portion 180 in an effort to increase axial rigidity of the frame portion 110.

As mentioned above, the tubular body portion 111 of the frame portion 110 may comprise lattice structures 121 that extend between each adjacent pair of the posts 180. The lattice structures 1221 also extend from the proximal end 114 to the distal end 115. In the exemplified embodiment, the lattice structures 121 and the posts 120 are integrally formed as a unitary structure free of seams. Thus, the tubular body portion 111 is a unitary/monolithic structure. In some embodiments, the tubular body portion 111 may be made from wire or may be laser cut from a tube, sheath, or the like. In the exemplified embodiment, the lattice structures 121 define a plurality of open cells 122 formed by intersecting struts 123. In the exemplified embodiment, all of the open cells 122 within all of the lattice structures 121 are diamond-shaped or partially diamond-shaped. The invention, however, is not to be so limited in all embodiments and other shapes for the open cells 122 are possible in other embodiments.

The frame portion 110 may further comprise a plurality of tabs 130 that protrude from the proximal end 114 of the tubular body portion 111 of the frame portion 110. In the exemplified embodiment, there are three of the tabs 130. However, the exact number of the tabs 130 is not to be limiting of the present invention in all embodiments and in other embodiments it may be possible to have just a single tab, or two tabs, or more than three tabs. The plurality of tabs 130 are circumferentially spaced apart along the circumference of the tubular body portion 111 of the frame portion 110. In the exemplified embodiment, the tabs 130 are equidistantly spaced apart along the circumference of the tubular body portion 111, although this is not required in all embodiments and different spacing arrangements may be used in other embodiments.

The tabs 130 may comprise a stem portion 131 that extends from the proximal end 114 of the tubular body portion 111 and an engagement portion 135 that is coupled to a terminal end of the stem portion 131. In the exemplified embodiment, the stem portion 131 comprises a first stem arm 132 and a second stem arm 133 that extend from the proximal end 114 of the tubular body portion 111. In alternative embodiments, a single stem arm may be used instead of two stem arms, and in other embodiments more than two stem arms may be included. In the exemplified embodiment, each of the tabs 130 is aligned with one of the posts 120 of the tubular body portion 111. Thus, in the exemplified embodiment the first and second arms 132. 133 of each of the stem portions 131 extends from the proximal end 114 of the tubular body portion 111 at a position that is aligned with one of the posts 120 (stated another way, the tabs 130 protrude from the posts 120 at the proximal end 114 of the tubular body portion 111 of the frame portion 110). In the exemplified embodiment, the first and second arms 132, 133 are spaced apart to define a diamond-shaped opening. In other embodiments, openings between the first and second arms 132, 133 may have other shapes.

The engagement portion 135 of the tabs 130 may extend from an end of the stem portion 131 which is furthest from the proximal end 114 of the tubular body portion 111. In the exemplified embodiment, the engagement portion 135 is oval shaped and elongated in a direction that is transverse to the axis A-A. In some embodiments, the engagement portion 135 may have a height (measured in a direction of the axis A-A) of about 1.6 mm (the term “about” as used herein including plus or minus 0.1 mm) and a width (measured in a direction perpendicular to the axis A-A) of about 2.5 mm (the term “about” as used herein including plus or minus 0.1 mm). The underside of the engagement portion 135 which faces the proximal end 114 of the tubular body portion 111 may form an engagement feature that serves to facilitate a coupling between the prosthetic valve 100 and the catheter apparatus 200 as described in more detail below. In the exemplified embodiment, the engagement portion 135 of the tabs 130 does not have any holes or apertures therein, although in other embodiments holes or apertures may be formed in the engagement portion 135.

In the exemplified embodiments, there are two sizes for the frame portion 110 of the prosthetic valve 100 with different heights and diameters for the frame portion 110 and for the tab portions 130. Of course, the frame portion 110 of the prosthetic valve 100 may be formed in other sizes as well. The tubular body portion 111 of the frame portion 110 comprises a first height H1 measured from the proximal end 114 of the tubular body portion 111 to the distal end 115 of the tubular body portion 111. Furthermore, the tabs 130 of the frame portion 110 comprise a second height H2 measured from the proximal end 114 of the tubular body portion 111 to a terminal end 136 of the tab 130.

In a first embodiment, the frame portion 110 has an outer diameter which is approximately 25 mm (the term “approximately” in this instance including plus or minus 1 mm). In the first embodiment, the first height H1 may be between 18 mm and 23 mm, more specifically between 19 mm and 22 mm, more specifically between 20 mm and 21 mm, and still more specifically approximately 20.6 mm (the term approximately in this instance including a plus/minus difference of 0.3 mm). In the first embodiment, the second height H2 may be between 3.4 mm and 4.0 mm, more specifically between 3.5 mm and 3.9 mm, more specifically between 3.6 mm and 3.8 mm, and still more specifically approximately 3.7 mm (the term approximately in this instance including a plus/minus difference of 0.05 mm). Thus, in the first embodiment a ratio of the first height H1 to the second height H2 may be in a range of 5.2:1 and 5.8:1, or more specifically a range of 5.3:1 and 5.7:1. or still more specifically 5.4:1 and 5.6:1.

In a second embodiment, the frame portion 110 has an outer diameter which is approximately 28 mm (the term “approximately” in this instance including plus or minus 1 mm). In the second embodiment, the first height HI may be between 21 mm and 27 mm, more specifically between 22 mm and 26 mm, more specifically between 23 mm and 25 mm, and still more specifically approximately 24 mm (the term approximately in this instance including a plus/minus difference of 0.3 mm). In the second embodiment, the second height H2 may be between 3.5 mm and 4.1 mm, more specifically between 3.6 mm and 4.0 mm, more specifically between 3.7 mm and 3.9 mm, and still more specifically approximately 3.8 mm (the term approximately in this instance including a plus/minus difference of 0.05 mm). Thus, in the second embodiment a ratio of the first height H1 to the second height H2 may be in a range of 6.2:1:1 and 6.6:1, or more specifically a range of 6.3:1 and 6.5:1. In some embodiments, the frame portion 110 may be configured so that a ratio of the first height H1 to the second height H2 is between 5.2:1 and 6.6:1.

In some embodiments, the stem portions 131 may have a height between 2 mm and 2.2 mm. In some embodiments, when the frame portion 110 has an outer diameter of 25 mm the stem portion 131 may have a height of approximately 2.05 mm and when the frame portion 110 has an outer diameter of 28 mm the stem portion 131 may have a height of approximately 2.15 mm (the term approximately in this instance including plus or minus 0.05 mm). In both exemplary embodiments. the engagement portions 135 may have a height of between 1.6 mm and 1.7 mm, and more specifically approximately 1.66 mm.

It should be noted that the frame portion 110 may be formed with other diameters and the examples described herein are intended to be exemplary. In some embodiments, the frame portion 110 may have an outer diameter of 22 mm. In some embodiments, the frame portion 110 may have an outer diameter of 25 mm. In some embodiments, the frame portion 110 may have an outer diameter of 28 mm. In some embodiments, the frame portion 110 may have an outer diameter of 31 mm.

Thus, in both of the embodiments noted herein, the tabs 130 have a length which is significantly less than a length of the tubular body portion 111 of the frame portion 110. This may allow for repositioning, retrieving, and/or recapturing of the prosthetic valve 100 during an installation procedure. That is, as will be described in greater detail below, when the prosthetic valve 100 is being deployed from the catheter apparatus 200 and installed in a patient, the tabs 130 are the last part of the prosthetic valve 100 that remain coupled to or engaged with the catheter apparatus 200. Because the overall height of the tubular body portion 111 of the frame portion 110 is quite small in comparison to other prosthetic aortic valves, the tubular body portion 111 may still be capable of being recaptured, retrieved, and/or repositioned even when only the tabs 130 remain engaged with the catheter apparatus 200. This provides the physician with a great deal of flexibility during the installation procedure and a great deal of confidence that the prosthetic valve 100 will be installed in the appropriate location within the patient.

The valve portion 180 may be cinched at the locations of the posts 120 and the tabs 130. The valve portion 180 may be coupled to the frame portion 110 at the locations of the posts 120 and the tabs 130 with the use of a commissure strip or the like.

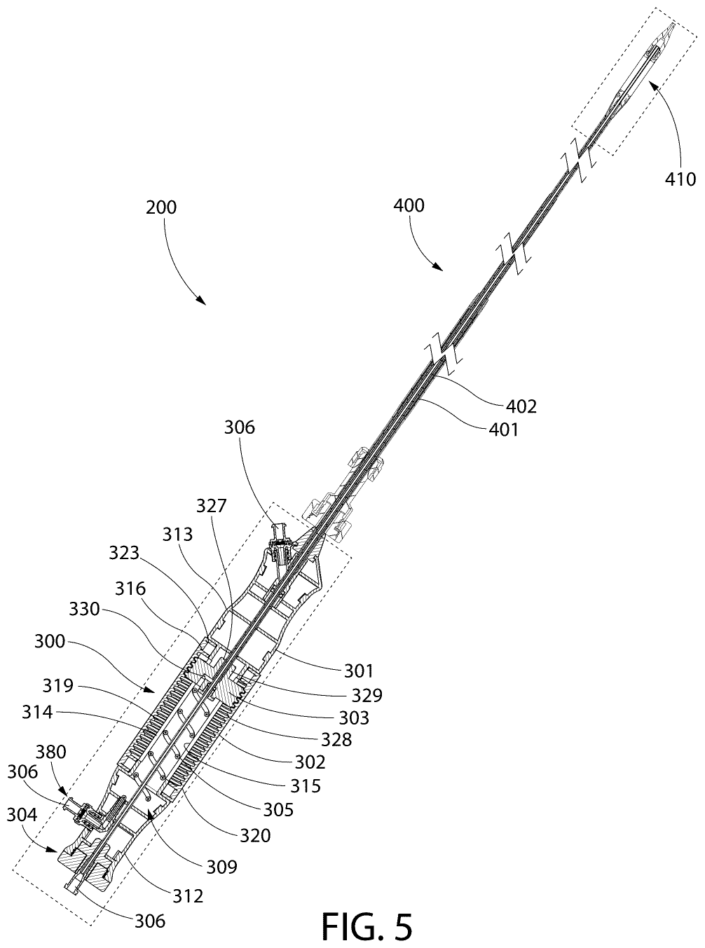

Referring to FIGS. 4 and 5, the catheter apparatus 200 will be described. The catheter apparatus 200 is utilized to install or implant the prosthetic valve 100 described above (and potentially also other alternative prosthetic valves) into a patient to replace a damaged aortic valve of the patient. The catheter apparatus 200 generally comprises a handle assembly 300 and a catheter assembly 400. The handle assembly 300 will be described in greater detail below with reference to FIGS. 4-8 and the catheter assembly 400 will be described in greater detail below with reference to FIGS. 4. 5, and 7-9. Generally, the handle assembly 300 is used to control the catheter assembly 400 such that actuation of the handle assembly 300 can be used to deploy a prosthetic aortic valve (such as the prosthetic valve 200) into a patient. The catheter assembly 400 comprises an outer shaft 401, an inner shaft 402, and a deployment assembly 410, the details of which will be described below. The outer and inner shafts 401, 402 may be collectively referred to as a shaft assembly in some embodiments.

Referring to FIGS. 4-8, the handle assembly 300 will be described in detail. The handle assembly 300 may comprise a handle housing 301, a rotating member 302, a slider 303, a proximal cap assembly 304, a flushing tube 305, and a plurality of flushing ports 306 that are used to flush out air from within and between the various components of the catheter assembly 400 when the catheter apparatus 200 is fully assembled by coupling the catheter assembly 400 to the handle assembly 300.

In the exemplified embodiment, the handle housing 301 may comprise first and second handle parts 307, 308, that are coupled together to define a handle cavity 309. The first handle part 307 has first connection features (e.g., snap-fit protrusions) 310 that engage with second connection features (e.g., apertures) 311 of the second handle part 308 to couple the first and second handle parts 307, 308 together. Of course, the first connection features 310 could be apertures and the second connection features 311 could be protrusions in other embodiments. Moreover, the handle housing 301 may be formed from a single part rather than two parts in other embodiments or may be coupled together in other ways, including friction or press fit, welding, fasteners such as screws, combinations thereof, or other known attachment techniques.

The handle housing 301 may comprise a proximal portion 312, a distal portion 313, and a central portion 314 located between the proximal and distal portions 312, 313. Furthermore, the handle housing 301 may comprise an inner surface 315 that defines the handle cavity 309 and an outer surface 316. The central portion 314 may have a reduced diameter as compared with the proximal and distal portions 312, 313. Thus, the central portion 314 may be recessed relative to the proximal and distal portions 312, 313 along the outer surface 316 of the handle housing 301. However, the invention is not to be so limited in all embodiments and the central portion 314 may not be recessed relative to the remainder of the handle housing 301 in all embodiments.

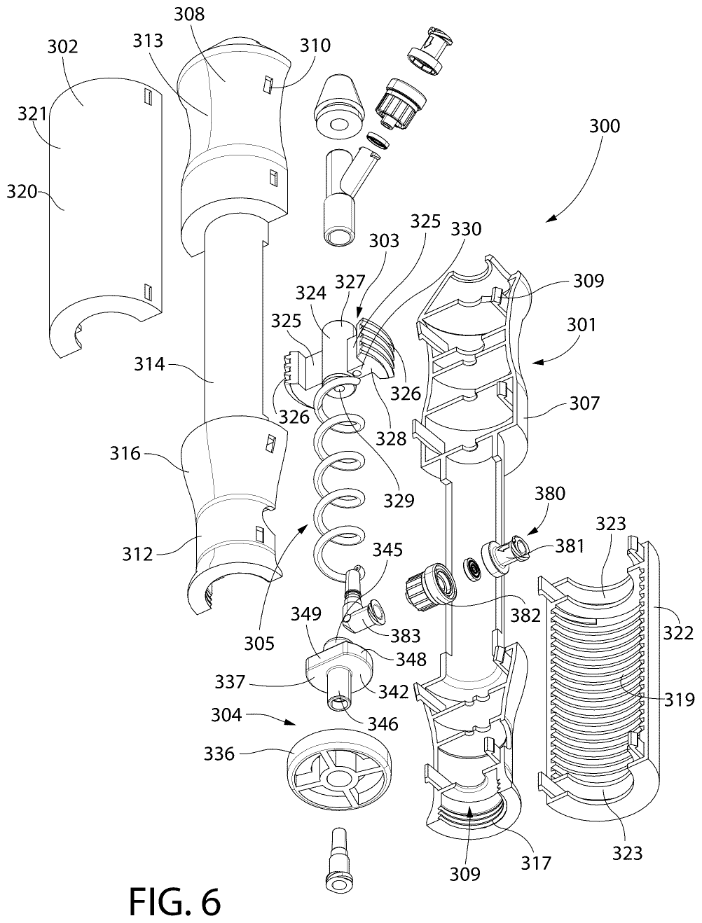

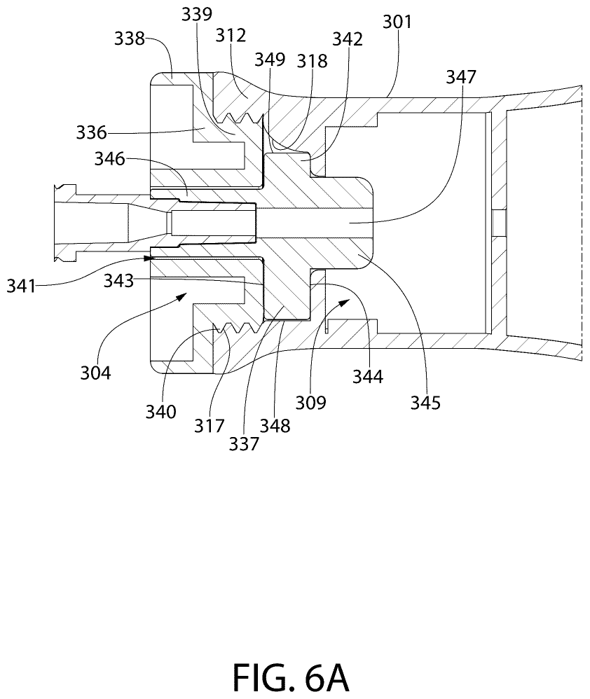

In the exemplified embodiment, the inner surface 315 of the proximal portion 312 of the handle housing 301 comprises a threaded portion 317 that is configured to engage with the proximal cap assembly 304 to couple the proximal cap assembly 304 to the handle housing 301. In the exemplified embodiment, the threaded portion 317 is located at the proximal-most end portion of the proximal portion 312 of the handle housing 301, although other locations for the threaded portion 317 may be permissible in other embodiments. Furthermore, the inner surface 315 of the proximal portion 312 of the handle housing 301 may comprise a first angular alignment feature 318, which is best shown in FIG. 6A. The first angular alignment feature 318 is an angled wall in the exemplified embodiment and it is configured to require the proximal cap assembly 304 to be oriented at a specific angular orientation in order for it to be inserted into the handle cavity 309. One purpose of this is to properly orient certain features of the catheter assembly 400 which may be coupled to the proximal cap assembly 304 as will be described in greater detail below. These features of the proximal portion 312 of the handle housing 301 will be discussed in some more detail below during the detailed description of the proximal cap assembly 304.

The rotating member 302 of the handle assembly 300 comprises a threaded inner surface 319 and an outer surface 320. The rotating member 302 of the handle assembly 300 is positioned around the central portion 314 of the handle housing 301 of the handle assembly 300. In the exemplified embodiment the rotating member 302 of the handle assembly 300 comprises a first rotating handle part 321 and a second rotating handle part 322 that are snap-fit connected together using snap-fit features. Of course, other connection techniques may be used including welding. fasteners, adhesives, or the like. Moreover, the rotating member 302 may be a singular part rather than two parts in other embodiments. The first and second rotating handle parts 321, 322 may be positioned around the central portion 314 of the handle housing 301 and then snap-fit connected together. The rotating member 302 may comprise ribs 323 that protrude from the threaded inner surface 319. When the rotating member 302 is positioned around the central portion 314 of the handle housing 301, the ribs 323 may contact the outer surface 316 of the handle housing 301 along the central portion 314 of the handle housing 301 and the threaded inner surface 319 of the rotating member 302 may be spaced apart from the outer surface 316 of the handle housing 310. This allows the threaded inner surface 319 of the rotating member 302 to engage the slider 303 to cause the slider to move axially as the rotating member 302 rotates relative to the handle housing 301, as discussed below.

The slider 303 may comprise a hub portion 324 that is disposed within the handle cavity 309 along the central portion 314 of the handle housing 301 and an engagement portion 325 that extends from the hub portion 324 and terminates in a threaded outer surface 326. The engagement portion 325 may comprise an arm that extends from the hub portion 324 and an arcuate threaded portion that terminates in the threaded outer surface 326. The threaded outer surface 326 may be arcuate, and more specifically convex, to facilitate the mating interaction between the threaded outer surface 326 of the slider 303 and the threaded inner surface 319 of the rotating member 302. A portion of the engagement portion 325 which includes the threaded outer surface 326 may be located outside of the handle cavity 309 in the space between the threaded inner surface 319 of the rotating member 302 and the outer surface 316 of the central portion 314 of the handle housing 301. In the exemplified embodiment, there are two engagement portions 325 located on opposite sides of the hub portion 324, although the invention is not to be so limited and the engagement portion 325 may be a continuous annular structure in other embodiments or there may just be a single engagement portion 325 that is semi-annular.

The slider 303 is positioned within the handle cavity 309 so that the threaded outer surface 326 of the slider 303 is in threaded engagement with the threaded inner surface 319 of the rotating member 302. Thus, as the rotating member 302 rotates relative to the handle housing 301, the engagement of the inner threaded surface 319 of the rotating member 302 with the threaded outer surface 326 of the slider 303 causes the slider 303 to move axially in a proximal axial direction (towards the proximal portion 312 of the handle housing 312) or a distal axial direction (towards the distal portion 313 of the handle housing 301), depending on the direction of rotation of the rotating member 302. For example, in one embodiment rotation of the rotating member 302 in a counterclockwise direction may cause the slider 303 to move in a proximal axial direction and rotation of the rotating member 302 in a counterclockwise direction may cause the slider 303 to move in a distal axial direction. In other embodiments, the opposite may be true such that counterclockwise rotation of the rotating member 302 moves the slider 303 in the distal axial direction and counterclockwise rotation of the rotating member 302 moves the slider 303 in the proximal axial direction. The rotation of the rotating member 302 of the handle assembly 300 and the resultant axial movement of the slider 303 controls movement of various components of the catheter apparatus 400 which may be coupled to the slider 303, as described in more detail below.

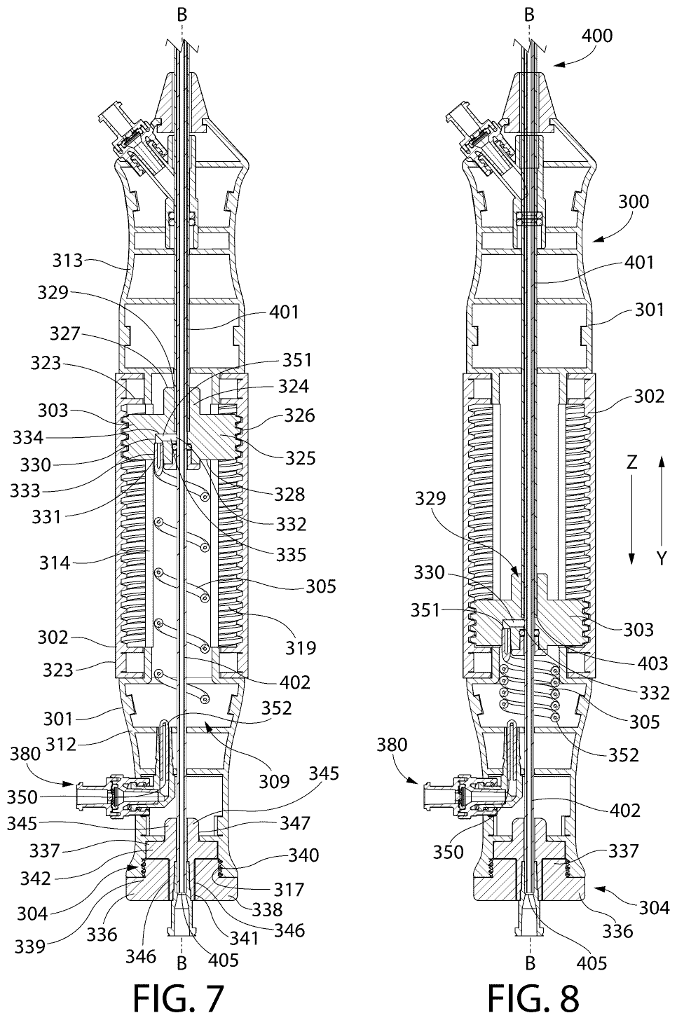

The slider 303 comprises a top surface 327 and a bottom surface 328. Furthermore, the slider 303 may comprise a through-hole 329 extending through the slider 303 in the axial direction from the top surface 327 to the bottom surface 328. In the exemplified embodiment, the through-hole 329 is located centrally along the slider 303 such that the through-hole 329 is formed through the hub portion 324 of the slider 303. The through-hole 329 forms a lumen in the slider 303 within which components of the catheter assembly 400 may be positioned as described below with reference to FIGS. 7 and 8. Briefly, the catheter assembly 400 comprises the outer shaft 401 and the inner shaft 402 that are at least partially located within the through-hole 329. The inner shaft 402 extends entirely through the through-hole 329 such that it protrudes from both the top and bottom surfaces 327, 328 of the slider 303. The outer shaft 401 terminates within the through-hole 329 and then protrudes from the top surface 327 of the slider 303, but the outer shaft 401 does not protrude from the bottom surface 328 of the slider 303. In some embodiments, the outer shaft 401 is coupled or attached or connected to the slider 303 so that movement of the slider 303 causes movement of the outer shaft 401. The inner shaft 402 may not be coupled to the slider 303 and as such movement of the slider 303 and the outer shaft 401 may be relative to the inner shaft 402.

The slider 303 may also comprise a flushing passageway 330 that extends from an opening 331 in the bottom surface 328 of the slider 303 to an outlet 332 that is fluidly coupled to the through-hole 329. The outlet 332 may be located internally within the slider 303 and not along any part of the outer surface of the slider 303. Rather, the outlet 332 may be positioned so as to ensure that any fluid introduced into the flushing passageway 330 will pass into the through-hole at a location between the top and bottom surfaces 327, 328 of the slider 303. In the exemplified embodiment, the flushing passageway 330 comprises a vertical portion 333 that extends from the opening (or inlet 331) to an elbow 334 and a horizontal portion 335 that extends from the elbow 334 to the outlet 332. Of course, the flushing passageway 330 may take on other pathway designs and configurations in other embodiments, including undulating paths, straight paths, linear paths, arcuate paths, or the like.

In the exemplified embodiment, the flushing passageway 330 is located at least partially within one of the engagement portions 325 of the slider 303. That is, in the exemplified embodiment the inlet 331 of the flushing passageway 330 is located along the engagement portion 325 of the slider, but the horizontal portion 335 of the flushing passageway 330 extends into the hub portion 324 of the slider 303 to place the flushing passageway 330 into fluid communication with the through-hole 329. In other embodiments, the flushing passageway 330 t could be located fully within the hub portion 324 of the slider 303 in other embodiments. Fluids introduced into the flushing passageway 330 through the opening 331 in the bottom surface 328 of the slider 303 may exit the flushing passageway 330 at the outlet 332 for introduction into the through-hole 329. Additional details about the usage and function of the flushing passageway 330, and in particular its relationship to the inner and outer shafts 401, 402 of the catheter assembly 400, will be described below with specific reference to FIGS. 7 and 8.

The proximal cap assembly 304 may comprise a proximal cap 336 and a proximal key 337. In the exemplified embodiment, the proximal cap 336 and the proximal key 337 are separate components that are coupled together, although in other embodiment the proximal cap 336 and the proximal key 337 may be integrally formed as a single component. The proximal cap 336 may comprise an end cap portion 338 and a threaded portion 339 that protrudes from a distal end of the end cap portion 338. The threaded portion 339 may comprise external threads 340 that are configured to mate with the threaded portion 317 of the proximal portion 312 of the handle housing 301, as best shown in FIG. 6A. Thus, the proximal cap 336 may be coupled to and detached from the handle housing 301 by engaging and disengaging the external threads 340 of the end cap portion 338 with the threaded portion 317 of the handle housing 301. The end cap portion 338 of the proximal cap assembly 304 may also comprise a through-hole 341 that extends therethrough from a distal end thereof to a proximal end thereof.

The proximal key 337 may comprise an angular alignment member 342 comprising a proximal end 343 and a distal end 344, a distal post 345 protruding from the distal end 344, and a proximal post 346 protruding from the proximal end 343. The proximal post 346 may nest within the through-hole 341 in the end cap portion 338 of the proximal cap assembly 304 to couple the proximal key 337 to the end cap portion 338. The distal post 345 may protrude through an opening formed in a wall that is an integral part of the handle housing 301. The coupling between the proximal key 337 and the end cap portion 338 may be a press-fit connection in some embodiments, although other types of connections may be used in other embodiments, including adhesives, fasteners, welding, or the like. The proximal key 337 and the end cap portion 338 could be a monolithic component in other embodiments. The proximal key 337 may comprise a through-hole 347 that extends through each of the distal post 345, the angular alignment member 342, and the proximal post 346.

The angular alignment member 342 may comprise an outer surface that extends between the proximal and distal ends 343, 344. The outer surface of the angular alignment member 342 may comprise a rounded portion 348 and a flat portion 349. In the exemplified embodiment, the proximal key 337 is only capable of being inserted into the proximal portion 312 of the handle housing 301 when the flat portion 349 of the outer surface is angularly (or rotationally) aligned with the first angular alignment feature 318 of the handle housing 301. As can be seen from FIG. 6A, the rounded portion 348 of the outer surface of the angular alignment member 342 will not fit in the area of the handle cavity 309 where the angular alignment feature 318 is located. Thus, the flat portion 349 of the outer surface of the angular alignment member 342 of the proximal key 337 forms a second angular alignment feature that mates with the first angular alignment feature 318 of the handle housing 301 to properly angularly orient/align the proximal key 337 within and relative to the handle housing 301. Of course, other features may be used to properly angularly orient the proximal key 337, such as different protrusions, recesses, ribs, ridges, channels, or the like. As will be discussed below, the proximal key 337 may be coupled (perhaps fixedly coupled) to the inner shaft 402 of the catheter assembly 400 and as such the angular alignment of the proximal key 337 relative to the handle housing 301 may also serve to properly angularly orient the inner shaft 402 and any components coupled thereto (such as the pusher, described further below), relative to the handle housing 301.

As mentioned above, there are a plurality of flushing ports 306 included as part of the handle assembly 300. One of the flushing ports 306 comprises an inlet port 380 which may comprise a check valve 381, a connector 382, and an elbow luer 383, although fewer components may make up the inlet port 380 in other embodiments. The inlet port 380 defines a passageway from a location external to the handle cavity 309 into the handle cavity 309 for purposes of flushing in between the inner and outer shafts 401, 402 of the catheter assembly 400, as discussed further below.

As noted above, the handle assembly 300 also comprises the flushing tube 305. The flushing tube 305 may comprise a first end 350, a second end 351, and a lumen 352 extending from the first end 350 to the second end 351. The lumen 352 is a flow channel that extends through the full length of the flushing tube 305 from the first end 350 to the second end 351. The first end 350 of the flushing tube 305 may be coupled to the inlet port 380. The second end 351 of the flushing tube 305 may be coupled to the slider 303, and more specifically to the flushing passageway 330 of the slider 303. Thus, fluid introduced into the inlet port 380 will pass into the lumen 352 of the flushing tube 305, through the second end 351 of the flushing tube 305 and into the flushing passageway 330 of the slider 303. This fluid is then introduced into the space between the inner and outer shafts 401, 402 of the catheter assembly 400 within the through-hole 329 in the slider 303, as will be discussed in greater detail below. The flushing tube 305 may be formed from polyvinyl chloride (PVC) tubing or any other type of flexible tubing as should be appreciated based on the operation of the flushing tube 305 as described further below.

In the exemplified embodiment, the flushing tube 305 has a coiled configuration. That is, the flushing tube 305 is arranged in a coiled configuration as it extends from the first end 350 to the second end 351. The flushing tube 305 has a pitch, which is the distance from the center of one coil to the center of an adjacent coil. Due to its coiled configuration, the flushing tube 305 is capable of compressing and expanding (like a compression spring) as the slider 303 moves axially within the handle cavity 309.

In particular, referring to FIGS. 7 and 8, the altering of the flushing tube 305 as the slider 303 moves axially within the handle cavity 309 is illustrated. In FIG. 7, the slider 303 is in its distal-most position (non-deployed position or non-deployed state of the catheter apparatus 200) and the flushing tube 305 is in its expanded configuration. As noted above, rotating the rotating member 302 of the handle assembly 300 in a first rotational direction will cause the slider 303 to move in a proximal axial direction, indicated by the arrow Z. Because the slider 303 is coupled to the second end 351 of the flushing tube 305, as the slider 303 moves in the proximal axial direction Z, the slider 303 causes the second end 351 of the flushing tube 305 to move in the proximal axial direction Z, thereby causing the flushing tube 305 to become compressed. By this, it is not meant that the tubing itself becomes compressed such that the diameter of the lumen 352 of the flushing tube 305 decreases. Rather, the coiled configuration of the flushing tube 305 compresses such that the pitch of the flushing tube 305 coils decreases. Similarly, as the slider 303 moves in the distal axial direction Y, the slider 303 and the second end 351 of the flushing tube 305 move in the distal axial direction Y, thereby causing the flushing tube 305 to expand from the configuration shown in FIG. 8 to the configuration shown in FIG. 7. Again, the expansion increases the pitch of the flushing tube 305 coils and does not affect the diameter or cross-sectional area of the lumen 352 of the flushing tube 305. Compression of the flushing tube 305 therefore decreases the axial length of the flushing tube measured between the first and second ends 350, 351 of the flushing tube 305. Furthermore, as the slider 303 moves in the proximal axial direction Z, the pitch of the flushing tube 305 decreases and as the slider 303 moves in the distal axial direction Y, the axial length and the pitch of the flushing tube 305 increases. Due to the coupling of the flushing tube 305 to the inlet port 380 and to the slider 303, the flushing tube 305 remains fluidly coupled to the inlet port 380 and the slider 303 (and more specifically, the flushing passageway 330) regardless of the axial position of the slider 303 within the handle cavity 309 so that flushing may take place at any axial position of the slider 303. The flushing tube 305 may be coupled to the inlet port 380 and to the slider 303 via a press fit, adhesives, interlocking structures, or combinations thereof.

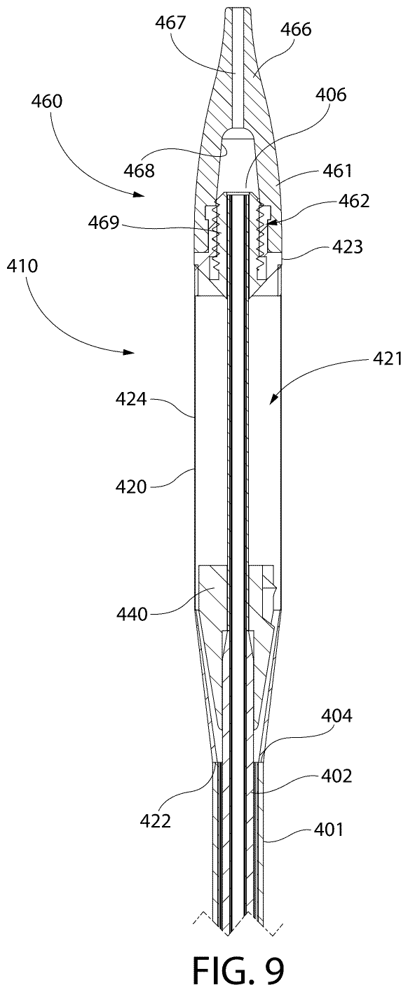

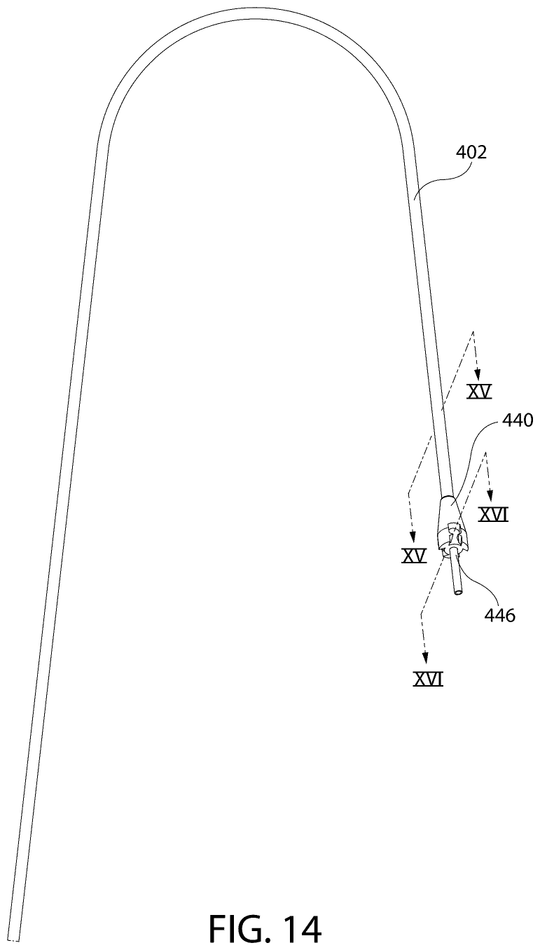

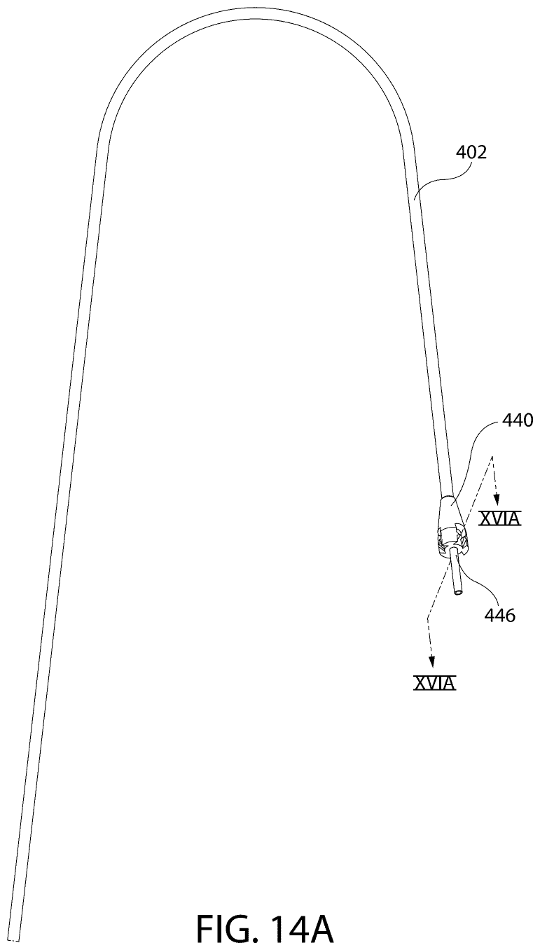

With continued reference to FIGS. 7 and 8, as noted above the catheter assembly 400 comprises an outer shaft 401 and an inner shaft 402. Each of the outer and inner shafts 401, 402 defines an interior passageway or lumen within which at least two axial wires extend. The axial wires may be flat wires or round wires and the two axial wires may be connected together in some embodiments. The axial wires (described in more detail below with reference to FIGS. 14A-16C) ensure that the outer and inner shafts 401, 402 bend in a particular manner and direction during use of the catheter apparatus 200. The inner shaft 402 is located within the interior passageway or lumen of the outer shaft 401 and the inner shaft 402 protrudes from/beyond the proximal and distal ends of the outer shaft 401. That is, the outer shaft 401 extends from a proximal end 403 shown in FIGS. 7 and 8 to a distal end 404 shown in FIG. 9. The inner shaft 402 may protrude or extend beyond each of the proximal 403 and distal ends 404 of the outer shaft 401.

The proximal end 403 of the outer shaft 401 is located within the through-hole 329 formed in the slider 303. More specifically, in the exemplified embodiment the proximal end 403 of the outer shaft 401 is located within the through-hole 329 of the slider 303 at a position that is immediately adjacent and distal to the outlet 332 of the flushing passageway 330 in the slider 303. The inner shaft 402 extends through the entirety of the through-hole 329 in the slider 303. The outer shaft 401 is axially aligned with a portion of the flushing passageway 330 such that an axis that is parallel to a longitudinal axis B-B of the handle assembly 300 intersects a portion of the outer shaft 401 and a portion of the flushing passageway 330. In the exemplified embodiment, the outlet 332 of the fluid passageway 330 abuts against the outer surface of the inner shaft 402 at a position immediately below (or proximal to) the outer shaft 401. Stated another way, the outlet 332 of the fluid passageway 330 is positioned radially inward of the outer shaft 401 so that the fluid introduced into the inlet port 380 of the handle assembly 300 flows between the inner and outer shafts 401, 402 for purposes of removing any air in the space between the inner and outer shafts 401, 402 and/or otherwise flushing the space between the inner and outer shafts 401, 402.

Moreover, the inner and outer shafts 401, 402 are not coupled together, but rather the outer shaft 401 is movable axially relative to the inner shaft 402 which is fixed axially, as described further below. Thus, there may be a small annular gap between the outer surface of the inner shaft 402 and the inner surface of the outer shaft 401. Thus, as the fluid exits the outlet 332 of the flushing passageway 330, the fluid may pass into the annular gap between the inner and outer shafts 401, 402. Due to the arrangement of the flushing tube 305, the space between the inner and outer shafts 401, 402 can be casily flushed regardless of the location or axial positioning of the slider 303 within the handle cavity 309.

Referring to FIGS. 7-10, the catheter assembly 400 will be further described. As noted above, the catheter assembly may comprise the outer shaft 401, the inner shaft 402, and the deployment assembly 410. The deployment assembly 410 comprises an outer sheath (or capsule) 420 that defines a sheath cavity 421, a pusher 440 that may be located within the sheath cavity 421, and a distal tip assembly 460 which may comprise a nose cone 461 and a distal thread assembly 462. The outer sheath 420 comprises a proximal end 422 and a distal end 423. The outer sheath 420 may be open at both of its proximal and distal ends 422, 423.

The outer shaft 402 may comprise the proximal end 403 that is coupled to the slider 303 of the handle assembly 300 and the distal end 404 that is coupled to the proximal end 422 of the outer sheath 420. While the proximal end distal ends 403, 404 are referred to as being coupled to the slider 303 and the outer sheath 420 respectively, in other embodiments other parts of the outer shaft 402 may be coupled to those components, such as locations between the proximal and distal ends 403, 404. Furthermore, as noted above, the slider 303 moves axially in the proximal and distal directions when the rotating member 302 of the handle assembly 300 is rotated. Thus, when the rotating member 302 is rotated in a first rotational direction, the slider 303 moves in the proximal axial direction Z. Furthermore, because the outer shaft 401 is coupled (or fixedly attached) to the slider 303, movement of the slider 303 in the proximal axial direction Z results in movement of the outer shaft 401 in the proximal axial direction Z. Finally, because the outer shaft 401 is coupled (or fixedly attached) to the outer sheath 420 of the deployment assembly 410 of the catheter assembly 400, as the slider 303 and the outer shaft 401 move in the proximal axial direction Z, the outer sheath 420 also moves in the proximal axial direction Z. Similarly, when the rotating member 302 of the handle assembly 300 is rotated in the opposite rotational direction, each of the slider 303, the outer shaft 401, and the outer sheath 420 moves in the distal axial direction Y.

In use, a prosthetic valve such as the prosthetic valve 100 described above may be positioned within the outer sheath 420 in the space 424 between the pusher 440 and the distal tip assembly 460. Thus, the pusher 440 and the distal tip assembly 460 may be spaced apart a sufficient axial distance to ensure that the prosthetic valve 100 will fit therein. This axial distance may be modifiable depending on the size of the prosthetic valve being used for a particular implantation operation. Movement of the outer shaft 420 in the proximal axial direction Z may cause the prosthetic valve 100 and the pusher 440 to become exposed so that the prosthetic valve 100 can be deployed and installed a patient, as described in more detail below. That is, movement of the outer shaft 420 in the proximal axial direction Z while the pusher 440 and the prosthetic valve 100 are stationary will cause the prosthetic valve 100, and then the pusher 440, to emerge from the opening in the distal end 423 of the outer sheath 420 for implantation or the like.

The inner shaft 402 of the catheter assembly 400 may comprise a proximal end 405 and a distal end 406. The pusher 440 may be fixedly coupled to the inner shaft 402 at a position that is adjacent to the distal end 406 of the inner shaft 402. That is, the pusher 440 may be located between the proximal and distal ends 405, 406 of the inner shaft 402, but in the exemplified embodiment the pusher 440 may be positioned much closer to the distal end 406 than to the proximal end 405. The pusher 440 may be fixed axially relative to the inner shaft 402 by any means desired, such as adhesive, welding, bonding, combinations thereof, or other technical means now known or later discovered. The pusher 440 may also be fixed rotationally relative to the inner shaft 402 such that the pusher 440 may be non-movable relative to the inner shaft 402.

In the exemplified embodiment, the proximal end 405 of the inner shaft 402 is coupled to the proximal cap assembly 304, and more specifically to the proximal key 337. However, the invention is not to be so limited in all embodiments and the proximal end 405 of the inner shaft 402 may be coupled to other parts of the handle assembly 300 in other embodiments, including being coupled directly to the handle housing 301 in some embodiments. Furthermore, it is possible that other portions of the inner shaft 402 than the proximal end 402 may be coupled to the handle assembly 300. In the exemplified embodiment, the distal end 406 of the inner shaft 402 is coupled to the distal tip assembly 460, and more specifically to the distal thread assembly 462 thereof. Other portions of the inner shaft 402 may be coupled to the distal tip assembly 460 in other embodiments. Furthermore, the distal end 406 of the inner shaft 402 may be coupled to other components of the distal tip assembly 460 in other embodiments. As noted above, the proximal cap assembly 304 may be threadedly and therefore fixedly (although detachably) coupled to the handle housing 301. By fixedly, it is meant that when the proximal cap assembly is coupled to the handle housing 301, the proximal cap assembly 304 is fixed such that it cannot move axially relative to the handle housing 301. However, due to the threaded attachment, the proximal cap assembly 304 may be entirely detached from the handle housing 301 and this will be discussed below with reference to the loading of the catheter apparatus 200 with the prosthetic valve 100. Because the inner shaft 402 is fixedly coupled to the proximal cap assembly 304, the angular orientation or positioning of the proximal cap assembly 304 due to the angular alignment features noted above may dictate the angular orientation or positioning of the inner shaft 402. This may help to ensure that the inner shaft 402 is oriented to bend in a proper direction during a valve replacement procedure.

In accordance with the exemplified embodiment, the inner shaft 402 is fixed axially between the proximal cap assembly 304 and the distal tip assembly 460. Thus, rotation of the rotating member 302 of the handle assembly 300 and the related axial movement of the slider 303, the outer shaft 401, and the outer sheath 420 is relative to the inner shaft 402 which is stationary and does not move axially or otherwise during the movement of the slider 303. Thus, as the outer sheath 420 moves in the proximal axial direction Z as noted above, the pusher 440 and the distal tip assembly 460 remain stationary due to their attachment to the inner shaft 402. As such, the pusher 440 becomes “unsheathed” or exposed and removed from the sheath cavity 421 once the outer sheath 420 moves a sufficient distance in the proximal axial direction Z. By coupling the inner shaft 402 to the proximal key 337 and including the angular orientation feature on the proximal key 337 and the handle housing 301, the proximal key 337 may serve to maintain the axial wires of the inner shaft 402 in alignment with the axial wires of the outer shaft 401 to ensure that both the inner and outer shafts 401, 402 curve in the same, desired direction during use.

As noted above, the pusher 440 may be coupled to the inner shaft 402 at a position that is near, but spaced apart from the distal end 406 of the inner shaft 402. In the exemplified embodiment, the distal thread assembly 462 comprises a distal thread component 463 and a cap component 464 that are coupled together. However, in other embodiments the distal thread component 463 and the cap component 464 may be an integral unitary structure. In the exemplified embodiment, the distal thread assembly 462 may be coupled to the inner shaft 402 at the distal end 406 of the inner shaft 402. That is, in the exemplified embodiment the inner shaft 402 may extend through a through-hole or passageway or lumen in the distal thread assembly 462 so that the distal end 406 of the inner shaft 402 is essentially flush with the distal end of the distal thread assembly 462. In other embodiments, the distal end 406 of the inner shaft 402 may be recessed relative to or slightly protrude from the distal end of the distal thread assembly 462. The distal thread component 463 comprises an outer threaded surface 465. The distal thread assembly 462, and more specifically the cap component 464 thereof, closes the distal end of the sheath cavity 421 when the outer sheath 420 is in a non-deployed or closed configuration, as shown in FIG. 9. When the outer sheath 420 is moved in the proximal axial direction Z as described herein, the distal thread assembly 462 remains stationary and therefore the distal end of the sheath cavity 421 is open which enables the prosthetic valve and the pusher 440 to exit the outer sheath 420 through the open distal end of the sheath cavity 421.

The distal tip assembly 460 may also comprise a nose cone 466. The nose cone 466 may comprise an inner surface 468 that defines an interior lumen 467. Furthermore, at least a portion of the inner surface 468 of the nose cone 466 is a threaded portion 469. Thus, the nose cone 466 may be detachably coupled to the distal thread component 463 via engagement between the threaded portion 469 of the nose cone 466 and the outer threaded surface 465 of the distal thread component 463. The interior lumen 467 may provide a passageway for fluid that is flushing the interior of the inner shaft 402 to exit the catheter assembly 400.

In FIG. 9, the two axial wires within each of the inner and outer shafts 401, 402 are visible as solid black lines extending within the lumens of the inner and outer shafts 401, 402. The intent is for the axial wires in the inner and outer shafts 401, 402 to remain in alignment to enable each of the inner and outer shafts 401, 402 to bend in the same direction during use, as mentioned above.

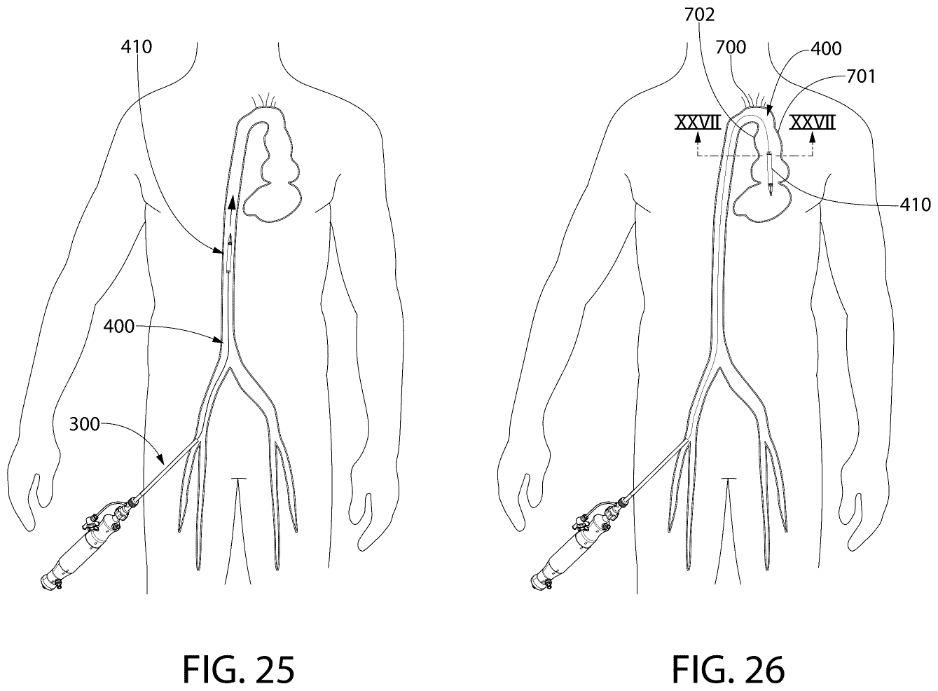

As noted above, during use when the rotating member 303 of the handle assembly 300 is rotated, the slider 303, the outer shaft 401, and the outer sheath 420 move in either the proximal or distal axial direction, depending on the direction of rotation of the rotating member 303. It is worth repeating that this movement is done relative to the inner shaft 402, the pusher 440, and the distal tip assembly 460, which remains stationary as the outer sheath 420 moves. Thus, when the slider 303 and the outer sheath 420 move in the proximal axial direction Z, because the pusher 440 and the distal tip assembly 460 remain stationary, any components previously located within the shaft cavity 421 exit therefrom and become exposed. In particular, a prosthetic valve 100 located in the axial space 424 between the pusher 440 and the distal tip assembly 460 becomes exposed and then the pusher 440 becomes exposed as the outer sheath 420 continues to move in the proximal axial direction Z. This will be described further below during the discussion of the valve deployment with reference to FIGS. 25-33.

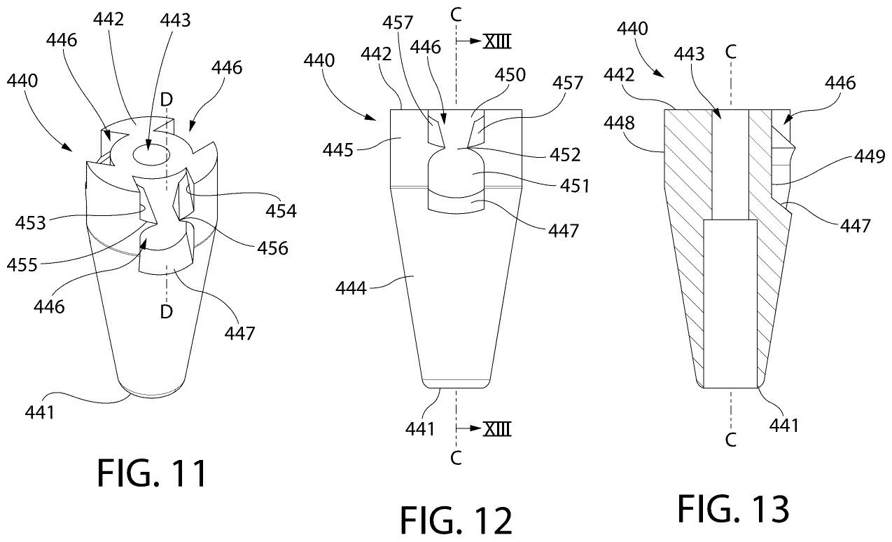

Referring to FIGS. 11-13, the pusher 440 will be further described. The pusher 440 is the component to which the prosthetic valve 100 is engaged or coupled prior to deploying and installing the prosthetic valve 100 within a patient. Thus, the pusher 440 may include features which engage or mate with features (e.g., the tabs 130) of the prosthetic valve 100 to facilitate this. Of course, the prosthetic valve 100 may not have the tabs 130 in all embodiments and thus other engagement between the pusher 440 and the prosthetic valve may be used in other embodiments. The pusher 440 may comprise a proximal end 441, a distal end 442, and a through-hole 443 extending from the proximal end 441 to the distal end 442. The inner shaft 402 may extend into and through the through-hole 443 for purposes of coupling or otherwise attaching the pusher 440 to the inner shaft 402.

In the exemplified embodiment, the pusher 440 comprises a cone-shaped proximal portion 444 and a cylindrical shaped distal portion 445. However, the invention is not to be so limited in all embodiments and the pusher 440 may be cone-shaped or cylindrical shaped or any other shape along its entire length in other embodiments. Furthermore, the pusher 440 may comprise at least one indentation (or recess or notch or channel) 446 that extends from the distal end 442 to a proximal end surface 447. In the exemplified embodiment, the pusher 440 comprises three of the indentations 446 that are arranged in a circumferentially and equidistantly spaced apart manner. However, the pusher 440 may have any number of the indentations 446 as may be desired. In certain embodiments, the number of the indentations 446 may correspond to the number of tabs 130 of the prosthetic valve 100 configured to be used with the catheter apparatus 200. The proximal end surface 447 is located distally of the proximal end 441 of the pusher 440 such that the indentations 446 do not extend the full length of the pusher 440. Furthermore, the indentations 446 are elongated in the axial direction between the proximal and distal ends 441, 442 of the pusher 440.

In the exemplified embodiment, the tabs 130 of the prosthetic valve 100 are equidistantly spaced apart by a distance of 120°. Similarly, the indentations 446 of the pusher 440 are equidistantly spaced apart by a distance of 120°. This provides symmetry and strength to the components. However, the invention is not limited by this equidistant spacing in all embodiments and in other embodiments the distances between the tabs 130 may not be the same for each pair of adjacent tabs, and the distances between the indentations 446 may not be the same for each pair of adjacent indentations.

Each of the indentations 446 forms a recess in an outer surface 448 of the pusher 440 that extends to a floor 449 that is recessed relative to the outer surface 448. The pusher 440 comprises a longitudinal axis C-C that extends between the proximal and distal ends 441, 442. In the exemplified embodiment, the proximal end surface 447 of the indentations 446 is sloped. More specifically, the proximal end surface 447 of the indentations 446 is sloped downwardly in a direction away from the distal end 442 and towards the proximal end 441 of the pusher 440 when moving in a direction from the floor 449 to the outer surface 448. The proximal end surface 447 is oriented at an oblique angle relative to the floor 449 and also relative to the longitudinal axis C-C.

Each of the indentations 446 may comprise an entry section 450 adjacent to the distal end 442 of the pusher 440, a nesting section 451 adjacent to the proximal end surface 447, and a neck section 452 located between the entry and nesting sections 450, 451. In the exemplified embodiment, the neck section 452 has a reduced width relative to the entry and nesting sections 450. 451. Furthermore, each of the indentations has a longitudinal axis D-D and a sidewall surface that extends from the floor 449 to the outer surface 448. The sidewall surface comprises a first portion 453 located on a first side of the longitudinal axis D-D and a second portion 454 located on a second side of the longitudinal axis D-D. Furthermore, each of the indentations 446 comprises a first tab 455 protruding from the first portion 453 of the sidewall surface and a second tab 456 protruding from the second portion 454 of the sidewall surface. The first and second tabs 455, 456 extend inwardly towards the longitudinal axis D-D to define the narrowed neck section 452 of the indentations 446. Furthermore, in the exemplified embodiment each of the first and second tabs 455. 456 has a distal surface 457 that faces the distal end 442 of the pusher 440. In the exemplified embodiment, the distal surfaces 457 of the first and second tabs 455, 456 are sloped downwardly in a direction away from the distal end 442 of the pusher 440.

As noted above and discussed in greater detail below, each of the indentations 446 may be configured to receive one of the tabs 130 of the prosthetic valve 100 described above. Thus, the prosthetic valve 100 may be generally positioned so that the proximal end 114 of the prosthetic valve 100 abuts against the distal end 442 of the pusher 440 and the tabs 130 of the prosthetic valve 100 nest within one of the indentations 446 of the pusher 440. The sloping of the proximal end surfaces 447 of the indentations 446 serves a functional purpose in accordance with embodiments of the present invention. In particular, if during deployment of the prosthetic valve 100 one of the tabs 130 becomes stuck within a respective one of the indentations 446, a user will be able to disengage the pusher 440 from the prosthetic valve 100 by moving the entire catheter apparatus 200 in a distal direction. Moving the catheter apparatus 200 in the distal direction will cause the proximal end surface 447 of the indentations 446 to move distally relative to the stuck prosthetic valve 100 so that the tab 130 which is stuck may slide over the sloped proximal end surface 447 to facilitate detachment of the prosthetic valve 100 from the pusher 440. If the proximal end surface 447 were oriented perpendicular to the longitudinal axis C-C, such movement of the pusher 440 relative to the prosthetic valve 100 would only cause the end of the tab 130 to abut against the proximal end surface 447. Thus, the sloping orientation of the proximal end surface 447 allows the tab 130 to easily glide over the proximal end surface 447 to ensure that the prosthetic valve 100 detaches from the pusher 440 to be deployed/installed into a patient.

Referring to FIGS. 14-16, the coupling of the pusher 440 to the inner shaft 402 of the catheter assembly 400 will be described in accordance with one embodiment of the present invention. As noted above, there are two axial wires 415, 416 located within the interior lumen of the inner shaft 402. The axial wires 415, 416 are positioned around an inner diameter polymer layer 418. The inner polymer layer (or liner) 418 may run the entire inner diameter length. The inner polymer layer 418 may provide lubricity and embed and seal the axial wires 415, 416 in the inner shaft 402. This inner polymer layer 418 may prevent damaging items passing through the internal diameter that could occur if the axial wires 415, 416 were exposed and not covered by the inner polymer layer 418.

The axial wires 415, 416 may be fixed relative to a sheath portion 417 of the inner shaft 402. Due to the axial wires 415, 416 being positioned within the interior lumen of the inner shaft 402, the inner shaft 402 is only able to bend in certain directions because the axial wires 415, 416 must be located one on top of the other during the bending. That is, attempting to bend the inner shaft 402 with the axial wires 415, 416 positioned in a side-by-side position (with one axial wire 415 adjacent to the inner curve and the other axial wire 416 adjacent to the outer curvature of the inner shaft 402) is not possible because the two axial wires 415, 416 will prevent such bending due to the combined strength of the axial wires 415, 416. Thus, prior to coupling the pusher 440 to the inner shaft 402, the inner shaft 402 is bent into a U-shaped bend configuration as shown in FIG. 14. When so bent, the first and second axial wires 415, 416 are aligned on a vertical plane E-E. The pusher 440 is then slid onto the inner shaft 402 while the inner shaft 402 is maintained in the U-shaped bend configuration. Finally, in one embodiment the pusher 440 may be rotated relative to the inner shaft 402 until one of the indentations 446 is aligned and/or centered on the vertical plane E-E.

Thus, with the inner shaft 402 in the U-shaped bend configuration, the pusher 440 may be coupled to the inner shaft 402 in a specific angular orientation to align the indentations 446 in a desired manner. In accordance with one embodiment of the present invention, it may be desirable to align one of the indentations 446 of the pusher 440 with the vertical plane E-E on which the two axial wires 415, 416 of the inner shaft 402 lie when the inner shaft 402 is in the U-shaped bend configuration. However, the invention is not to be limited to this angular orientation of the pusher 440 relative to the inner shaft 402 in all embodiments.

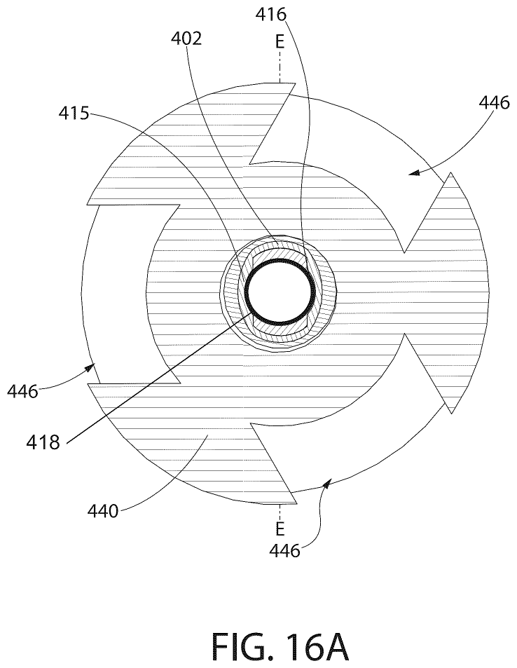

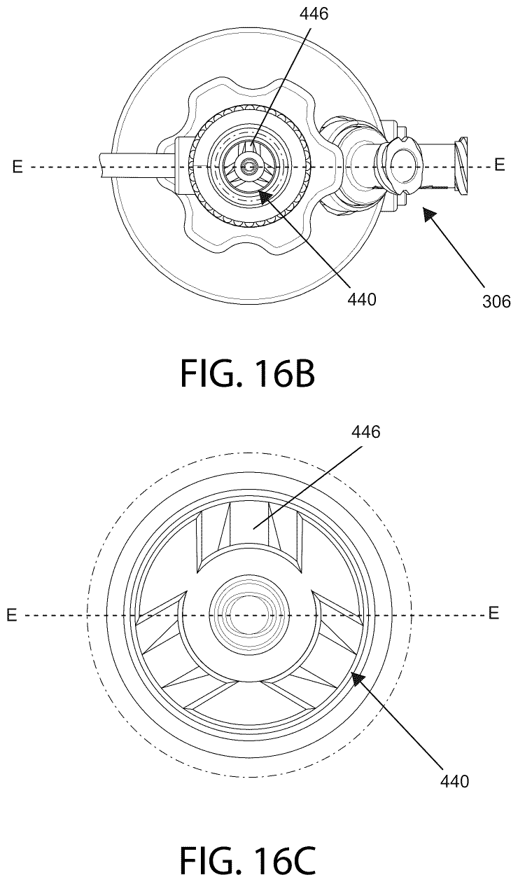

Referring to FIGS. 14A and 16A, in another embodiment, the pusher 440 may be rotated 90° relative to the angular orientation shown in FIGS. 14 and 16 so that the one of the plurality of indentations 446 is offset 90° from the vertical plane E-E. That is, rather than orienting the pusher 440 so that one of the indentations 446 is aligned with the vertical plane E-E, in this embodiment one of the indentations 446 is positioned between 80° and 100°, and more specifically approximately 90° offset from the vertical plane E-E. More specifically, one of the indentations 446 may be rotated 90° in the counterclockwise direction relative to the vertical plane E-E. In this embodiment an axis or plane F-F that is perpendicular to the vertical plane E-E intersects the one of the indentations 446. This embodiment, which is consistent with FIG. 28 described below, ensures that each of the indentations 446 (and hence also the tabs 130 if the prosthetic valve 100 located therein) is simultaneously at the furthest possible location away from the outer wall of the aortic arch during deployment of the prosthetic valve 100 into the patient. When viewed in a cross-section while the inner shaft 402 is in the bend configuration, one of the indentations 446 is at the 9 o'clock position, another one at the 1 o'clock position, and another one at the 5 o'clock position.

In the exemplified embodiment, when the inner shaft 402 (or the shaft assembly generally) is in the U-shaped bend configuration shown in FIG. 14A, the inner shaft 402 may have a first leg 480 and a second leg 481 that are aligned on the vertical plane E-E when the inner shaft 402 is in the U-shaped bend configuration. The outer surface of the inner shaft 402 may have a first circumferential portion 482 and a second circumferential portion 483. In the U-shaped bend configuration, the first circumferential portion 482 of the second leg 481 faces the first leg 480 and the second circumferential portion 483 of the second leg 481 faces away from the first leg 480. In this embodiment, the pusher 440 is coupled to the inner shaft 402 so that the one of the indentations 446 is located along the first circumferential portion 482 of the outer surface of the inner shaft 402. None of the indentations 446 is located along the second circumferential portion 482 of the inner shaft 402 at a position that is between 60° and 120°, or more specifically 80° and 100°, or more specifically approximately 90° offset from the vertical plane E-E.

Referring to FIGS. 16B and 16C, an embodiment which corresponds to the embodiment of FIGS. 14A and 16A is illustrated. In this embodiment, the vertical plane E-E is on the same plane as (and intersects) the flushing ports 306. Thus, when the handle is oriented laterally as shown in FIG. 16B, one of the indentations 446 of the pusher 440 is facing up (offset 90° relative to the vertical plane E-E of the axial wires).

The rotational orientation of the pusher 440 relative to the inner shaft 402 of the catheter assembly 400 may dictate the positioning of the indentations 446 of the pusher 440, and therefore also the tabs 130 of the prosthetic valve 100, relative to the aortic arch during a valve replacement procedure. Specifically, the aortic arch has an outer wall and an inner wall. In some instances, it has been found that when the last tab 130 of the prosthetic valve 100 to be released from the pusher 440 is positioned along the second circumferential portion 482 of the second leg 481 at a position approximately 90° offset from the vertical axis E-E (in a first rotational direction, and thereby positioned closest to the outer wall of the aortic arch), there is a greater likelihood that the tab 130 will become stuck (which may necessitate moving the catheter apparatus 200 distally as discussed above). Thus, there is a desire in some embodiments to keep all of the indentations 446 (and hence also the tabs 130 which are positioned within the indentations 446) as far away as possible from the outermost part of the second circumferential portion 483 of the outer surface of the shaft assembly, and hence also as far away as possible from the outer wall of the aortic arch during an implantation procedure. As noted above, due to the axial wires in the inner and outer shafts 401, 402, the bend direction of the catheter assembly 400 is known. Thus, in some embodiments the pusher 440 may be coupled to the inner shaft 402 in an angular orientation that ensures that each of the tabs 130 is positioned as far away from the outer wall of the aortic arch as possible. As mentioned above, this concept will be described in greater detail below with reference to FIGS. 26-28.

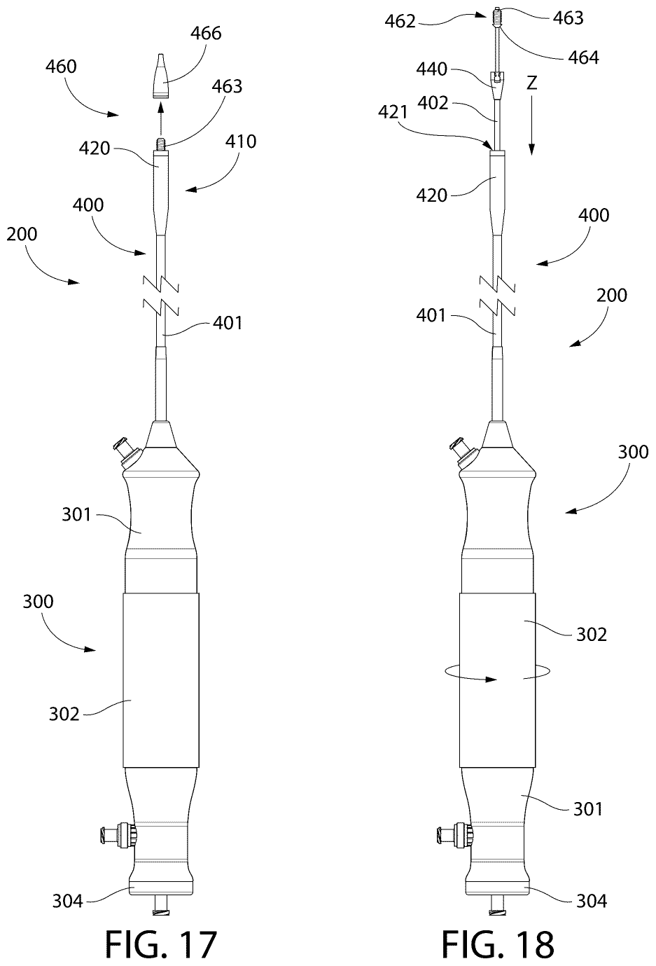

Referring to FIGS. 17-24 sequentially, the method and manner of loading the self-expanding prosthetic aortic valve 100 described above with reference to FIGS. 1-3 into the catheter apparatus described above with reference to FIGS. 4-16 will be described in accordance with an exemplary embodiment of the present invention.

Referring first to FIG. 17, the first step in the loading process is to detach the nose cone 466 from the distal thread component 463. This is achieved in the exemplified embodiment by unscrewing the nose cone 466 by rotating the nose cone 466 relative to the distal thread component 463 which is fixed due to its coupling to the inner shaft 402 (the inner shaft 402 is not visible in FIG. 17).

Next, referring to FIG. 18, once the nose cone 466 has been removed from the distal thread component 463, the user may rotate the rotating member 302 of the handle assembly 300 in a first rotational direction. In the exemplified embodiment, the first rotational direction is counterclockwise, although the invention is not to be so limited and in other embodiments the first rotational direction may be clockwise. As discussed above, rotating the rotating member 302 of the handle assembly 300 in the first rotational direction causes the slider 303, the outer shaft 401 and the outer sheath 420 to move in the proximal axial direction Z. As the rotating member 302 is rotated in the first rotational direction, the outer sheath 420 eventually moves a sufficient distance in the proximal axial direction Z such that the pusher 440 is entirely removed from the sheath cavity 421. Because the pusher 440 and the distal thread assembly 462 are fixed axially due to their coupling to the inner shaft 402, which is fixed axially during the axial movement of the slider 303, the outer shaft 401, and the outer sheath 420, as the outer sheath 420 moves in the proximal axial direction Z, a portion of the inner shaft 421, the pusher 440, and the distal thread component 462 become exposed as shown in FIG. 18.

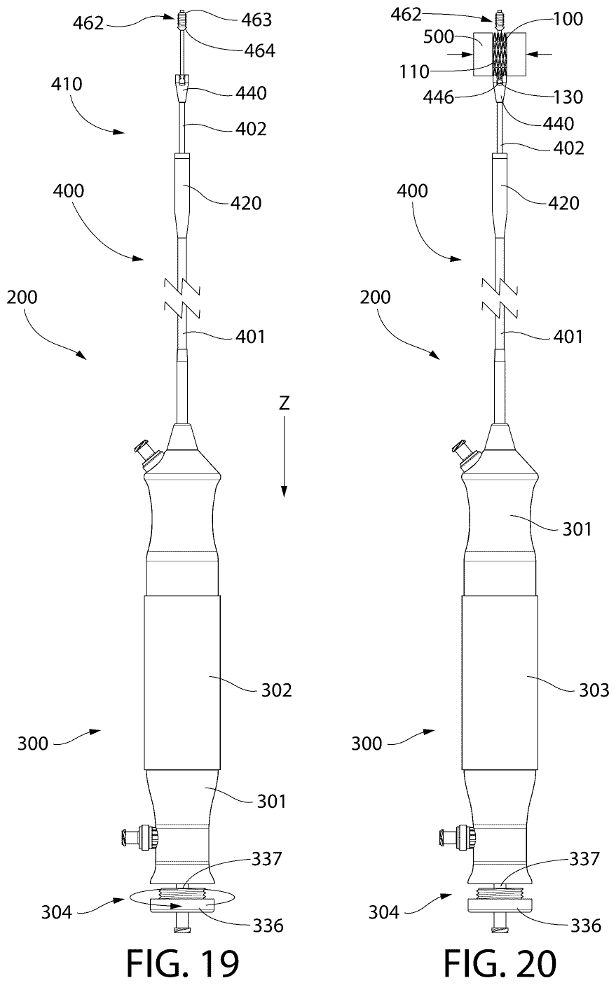

Next, referring to FIG. 19, the user may disengage the proximal cap assembly 304 from the handle housing 301. As discussed above, this is achieved in the exemplified embodiment by unscrewing the proximal cap 336 from the proximal end of the handle housing 301. Because the proximal key 337 is coupled to the proximal cap 336, the proximal key 337 will move in the proximal axial direction Z while the proximal cap 336 is unscrewed from the handle housing 301. As noted above, the proximal cap assembly 304 may be coupled to the proximal end 405 of the inner shaft 402. When the proximal cap assembly 304 is detached from the handle housing 301, the inner shaft 402 is free to move in the proximal axial direction Z relative to the handle housing 301. Specifically, a user could pull the proximal cap assembly 304 or push the distal thread assembly 462 in the proximal axial direction Z and it will move accordingly. It is the attachment of the proximal cap assembly 304 to the handle housing 301 which prevents the axial movement of the inner shaft 402 and the components coupled thereto (i.e., the pusher 440 and the distal thread assembly 462) when the catheter apparatus 200 is fully assembled.

Referring to FIG. 20, now that the pusher 440 has been exposed and the proximal cap assembly 304 has been decoupled from the handle housing 301, the catheter apparatus 200 is ready to be loaded with the prosthetic valve 100. To load the catheter apparatus 200 with the prosthetic valve, the prosthetic valve 100 is radially compressed by a radial compression apparatus 500. The radial compression apparatus 500 is illustrated generally in FIG. 20. It should be appreciated that the radial compression apparatus 500 may take any desired form and may include a variety of different apparatuses that arc configured to radially compress an article. For example, radial compression stations/machines/crimpers sold by Blockwise may be utilized for this purpose.

While the prosthetic valve 100 is being maintained in a radially compressed state, the prosthetic valve 100 is loaded onto the pusher 440. Specifically, in the exemplified embodiment loading of the prosthetic valve 100 onto the pusher 440 is achieved by aligning the tabs 130 of the frame portion 110 of the prosthetic valve 100 with the indentations 446 of the pusher 440. Once the tabs 130 are aligned with the indentations 446, the radial compression station 500 may further compress the prosthetic valve 100 to pop the tabs 130 into place within the indentations 446 of the pusher 440. The tabs 130 will be positioned within the indentations 446 so that the engagement portion 135 of the tabs 130 nests within the nesting section 451 of the indentations 446 and the stem 131 of the tabs 130 nests within the neck 452 and entry sections 450 of the indentations 446. Furthermore, the proximal end 114 of the prosthetic valve 100 may abut against the distal end 442 of the pusher 440. For features of the prosthetic valve 100 and the pusher 440 described in this paragraph but not labeled in FIG. 20 due to space constraints, reference may be made to FIGS. 2 and 12.

In some embodiments, the engagement of the tabs 130 of the prosthetic valve 100 with the indentations 446 of the pusher 440 may not be sufficient to maintain the prosthetic valve 100 in the radially compressed state. Rather, the prosthetic valve 100 may need to be positioned within the outer sheath 420 in order to maintain the prosthetic valve 100 in the radially compressed state upon disengagement of the prosthetic valve 100 from the radial compression station 500. Thus, even after the tabs 130 have nested within the indentations 446 of the pusher 440, the radial compression station 500 may maintain contact with the prosthetic valve 100 to maintain the prosthetic valve 100 in the radially compressed state as shown in FIG. 20.

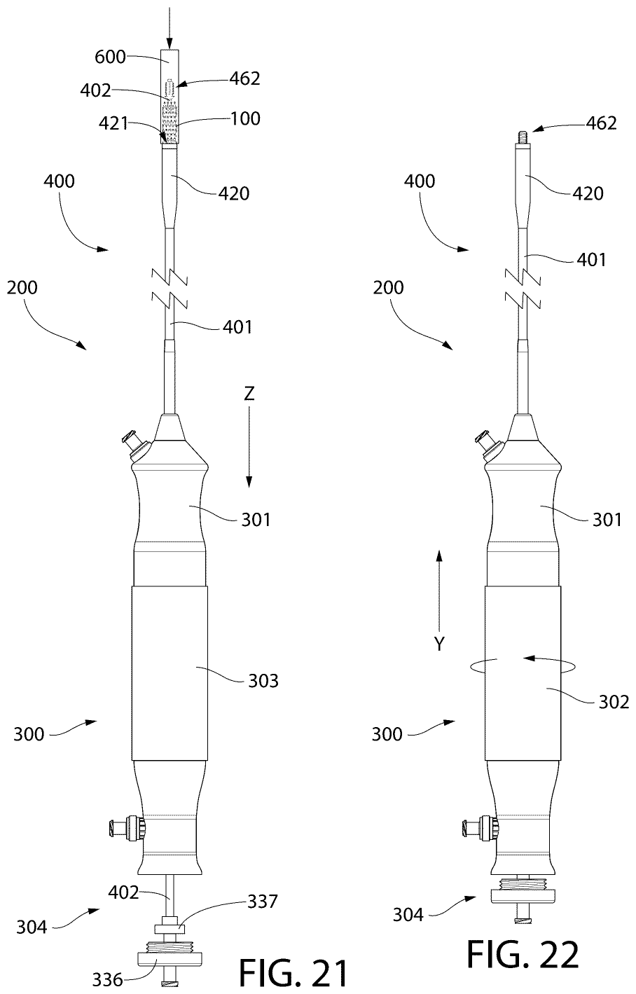

Next, referring to FIG. 21, while maintaining the prosthetic valve 100 in the radially compressed state, the user can push the prosthetic valve 100 in the proximal axial direction Z. In FIG. 21, the radial compression station 500 is not depicted, however, during the initial step of pushing the prosthetic valve 100 in the proximal axial direction Z, the radial compression station 500 may continue to engage with the prosthetic valve 100 to maintain the prosthetic valve 100 in the radially compressed state. The radial compression station 500 may maintain the prosthetic valve 100 in the radially compressed state until a certain percentage, for example 30%, or 40%, or 50%, or 60%, or 70%, or 80%, or 90%, or 100%, of the length of the prosthetic valve 100 is located within the sheath cavity 421 of the outer sheath 420. The pushing of the prosthetic valve 100 in the proximal axial direction Z may be done with a pushing tool 600, which is depicted generically in FIG. 21. The pushing tool 600 may have a cavity that can receive the distal thread assembly 462 to enable the pushing tool 600 to directly engage the prosthetic valve 100 to push it into the outer sheath 420. The user may use the pushing tool 600 to push the prosthetic valve 100 in the proximal axial direction Z by applying a force against the prosthetic valve 100 in the proximal axial direction Z. This will press the prosthetic valve 100 against the distal end of the pusher 440, which will force the pusher 440, the inner shaft 402, and the proximal cap assembly 304 to move in the proximal axial direction Z. Because the proximal cap assembly 304 was previously disengaged from the handle housing 301, applying a force onto the pusher 440 in the proximal axial direction Z will cause the pusher 440, the inner shaft 402, the distal thread assembly 462, and the proximal cap assembly 304 to move or slide or translate in the proximal axial direction Z. In FIG. 21, it can be seen that the proximal cap assembly 304 has moved in the proximal axial direction relative to its position in FIG. 20.

While the prosthetic valve 100, the pusher 440, and the proximal thread assembly 462 are moving in the proximal axial direction Z, the outer sheath 420 is stationary. Thus, the prosthetic valve 100, the pusher 440, and the proximal thread assembly 462 move in the proximal axial direction Z relative to the outer sheath 420 until the pusher 440, the prosthetic valve 100, and the distal thread assembly 462 begin to enter the sheath cavity 421 of the outer sheath 420. In FIG. 21, the pusher 440 is located entirely within the sheath cavity 421 (and is therefore not visible) and the prosthetic valve 100 is positioned partially within the sheath cavity 421. The user will continue to apply the force in the proximal axial direction Z onto the pusher 440 and/or the prosthetic valve 100 until the prosthetic valve 100 is entirely positioned within the sheath cavity 421. In some embodiments, the user force will be applied until the distal thread assembly 462 is located within or immediately distal to the sheath cavity 421. In some embodiments, the user applies the loading force onto the prosthetic valve 100 until there is a hard stop and the prosthetic valve 100 cannot be advanced any further, which may occur due to the pusher 440 bottoming out against the proximal interior surface of the outer sheath 420.

Referring to FIG. 22, once the prosthetic valve 100 is properly sheathed within the sheath cavity 421 of the outer sheath 420, the user may rotate the rotating member 302 of the handle assembly 300 relative to the handle housing 301 in a second rotational direction, which is the opposite rotational direction to that which was described with reference to FIG. 18. In the exemplified embodiment, the second rotational direction is clockwise, but as noted herein the first rotational direction may be clockwise and the second rotational direction may be counterclockwise in alternative embodiments. As may be appreciated, rotating the rotating member 302 of the handle assembly 300 in the second rotational direction causes the slider 303 (not visible in FIG. 22, but shown in FIGS. 7 and 8) to move in the distal axial direction Y. Furthermore, because the slider 303 is coupled to the outer shaft 401 of the catheter assembly 400, which is in turn coupled to the outer sheath 420, the outer shaft 401 and the outer sheath 420 also move in the distal axial direction Y. Finally, as the outer sheath 420 moves in the distal axial direction Y, the outer sheath 420 abuts against the distal thread component 462. As such, the movement of the outer sheath 420 in the distal axial direction Y causes the distal thread component 462 to move in the distal axial direction Y. Finally, because the distal thread component 462 is fixedly coupled to the inner shaft 402, the inner shaft 402, the pusher 440, and the prosthetic valve 100 also move in the distal axial direction Y as a result of the rotation of the rotating member 302 of the handle assembly 300. Thus, the rotation of the rotating member 302 of the handle assembly 300 in the clockwise direction at this stage in the loading process causes the proximal cap assembly 304, the outer shaft 401, the outer sheath 420, the pusher 440, the prosthetic valve 100, the inner shaft 402, and the distal thread assembly 462 to move the same distance in the distal axial direction Y. The rotating member 302 may be rotated in the second rotational direction until it comes to a hard stop and is no longer able to be rotated in the second rotational direction. This may occur as a result of the slider 303 abutting against an interior wall of the handle housing 301 as the slider 303 reaches its distal-most position, such as the position shown in FIG. 7.

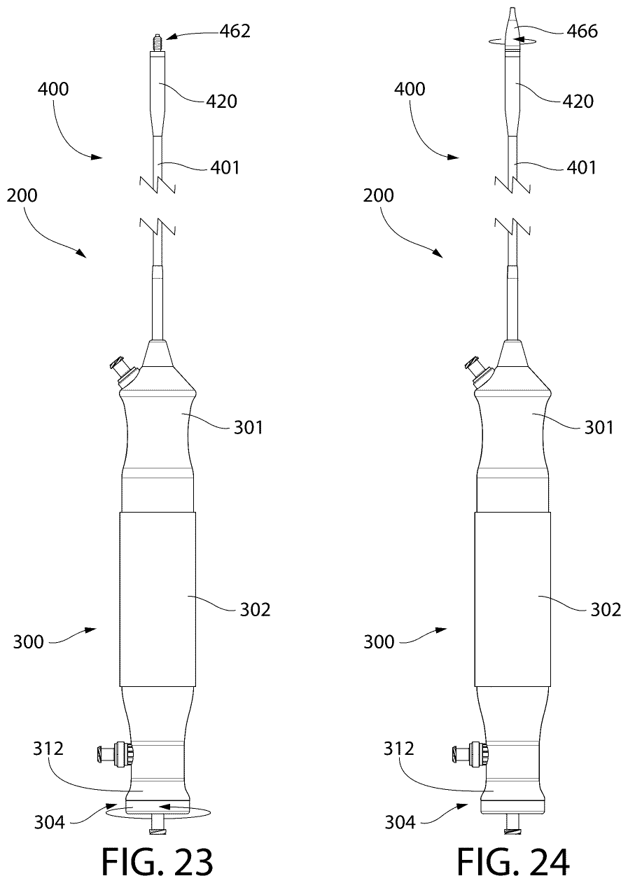

Next, referring to FIG. 23, the proximal cap assembly 304 is coupled to the handle housing 301. As described herein, in the exemplified embodiment this is achieved by engaging the screw threads of the proximal cap assembly 304 with the screw threads along the interior of the proximal portion 312 of the handle housing 301. It should be appreciated that screw threads is merely exemplary and other means may be used to detachably couple the proximal cap assembly 304 to the handle housing 301. For example, the proximal cap assembly 304 may be coupled to the handle housing 301 using a snap-fit arrangement, a tight fit, a set screw, or the like. Once the proximal cap assembly 304 is coupled to the handle housing 301, the distal thread assembly 462, the pusher 440, the inner shaft 401, and the proximal cap assembly 304 are axially fixed.

Finally, referring to FIG. 24, the nose cone 466 is coupled to the distal thread component 462. In the exemplified embodiment, the nose cone 466 is screwed onto the distal thread component 462, although other techniques may be used in other embodiments including tight fit, snap-fit, set screw, or the like. At this point, the prosthetic valve 100 is loaded within the outer sheath 420 and ready to be deployed and installed within a patient, the method of which will be described next.

The deployment of the prosthetic valve 100 into a patient body using the catheter apparatus 200 will be described with reference to FIGS. 25-33. Prior to the deployment and implantation of the prosthetic valve 100 into the patient body using the catheter apparatus 200, the catheter apparatus 200 is loaded with the prosthetic valve 100 as described herein.

Referring first to FIGS. 25 and 26, the first step in an aortic valve replacement procedure using the catheter apparatus 200 and the prosthetic valve 100 is to position the deployment assembly 410 of the catheter apparatus 200 in the proper position (i.e., at the implantation location) within the patient's body so that when the prosthetic valve 100 is deployed, it is located in the patient's aortic annulus. This is achieved by inserting the catheter apparatus 200 into an incision in the patient's body with a distal end of the catheter apparatus 200 entering the incision first (the distal end of the catheter apparatus 200 being formed by the distal end of the nose cone 466). The deployment assembly 410 may be continued to be moved into the patient body until the deployment assembly 410 is positioned at the location of the native aortic valve along the aortic arch. In the exemplified embodiment, the deployment assembly 410 is inserted into the patient's body through an incision along the leg and into the groin of the patient. In other embodiments the deployment assembly 410 may be inserted through an incision in the chest area or at other body locations. The deployment assembly 410 is then moved, by following a guide wire or using other techniques common in the industry, distally into the aortic arch of the patient. The surgeon may place the deployment assembly 410 of the catheter apparatus 200 so that the prosthetic valve 100 is located within a diseased aortic valve in the heart of the patient, as shown in FIG. 26.

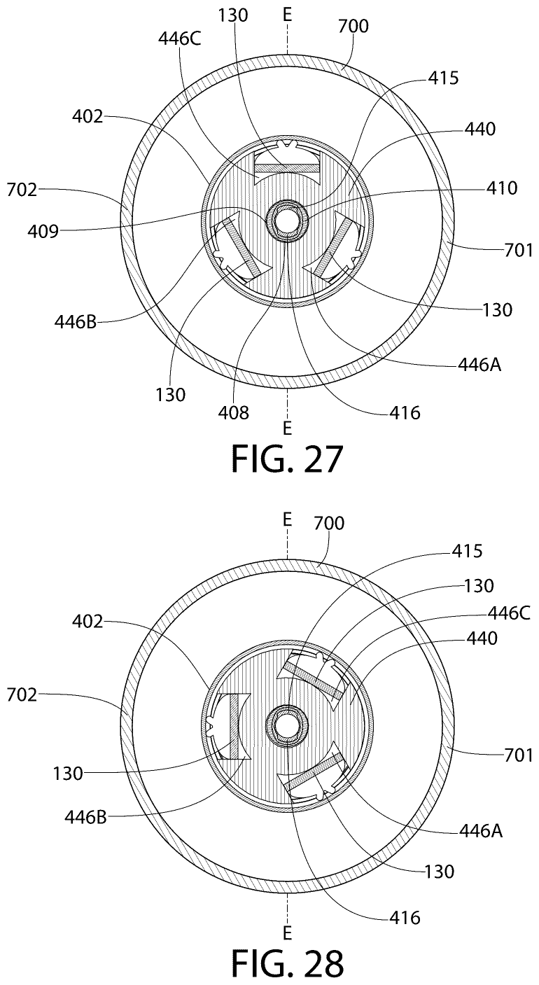

FIG. 27 is a cross-sectional view taken through the deployment assembly 410 at a location that is aligned with the pusher 440 while the deployment assembly 410 is located at the implantation location within the aortic arch annulus. Thus, a portion of the aortic arch 700 is also included in this view. The aortic arch 700 has an outer wall 701 and an inner wall 702. It has been determined through experimentation that the prosthetic valve 100 is most seamlessly deployed from the catheter apparatus 200 when none of the tabs 130 of the prosthetic valve 100 (and hence also none of the indentations 446 of the pusher 440) are positioned in the closest possible position relative to the outer wall 701 of the aortic arch 700 (i.e., at the 3 o'clock position in FIG. 27, with all references to a clock position being a position where the vertical plane E-E intersects the 12 and 6 o'clock positions). Thus, the pusher 440 is attached to the inner shaft 402 in such a manner to ensure this. As noted above with reference to FIGS. 14 and 16, in one exemplary embodiment, the pusher 440 is attached to the inner shaft 402 in an orientation with one of the indentations 446 aligned with the vertical plane E-E that intersects the axial wires 415, 416 located within the inner shaft 402. In such an orientation, one of the indentations 446a is not as far away as possible from the outer wall 701 of the aortic arch 700. However, it should be noted that the one of the indentations 446a is not directly facing and in the closest position to the outer wall 701, which would only occur if the pusher 440 were rotated counterclockwise between 30° so that the indentation 446a is positioned at the 3 o'clock position along the cross-section of the aortic arch 700. The indentation 446a is at the 4 o'clock position in FIG. 27.

FIG. 28 illustrates an alternative embodiment (based on the FIGS. 14A and 16A described above) whereby the pusher 440 has been rotated in the clockwise direction approximately 30° relative to that shown in FIG. 27 so that the indentation 446b is located at the 9 o'clock position and is aligned with the first circumferential portion 482 of the outer surface of the inner shaft 402 (which may equate to the indentation 446b directly facing the inner wall 702 of the aortic arch 700). When the indentation 446b is in the 9 o'clock position along the cross-section of the aortic arch 700 (and potentially directly facing the inner wall 702 of the aortic arch 700), the other indentations 446a, 446c are located as far away as possible from the outermost portion of the second circumferential portion 483 of the outer surface of the inner shaft 402 (and potentially also as far away as possible from the outer wall 701 of the aortic arch 700 that both of the other indentations 446a, 446c can be simultaneously). That is, any rotation of the pusher 440 relative to the inner shaft 402 from the position shown in FIG. 28 will move one of the indentations 446a, 446c further from the outermost portion of the second circumferential portion 483 (and potentially also from the outer wall 701 or the aortic arch) and the other one of the indentations 446a, 446c closer to the outermost portion of the second circumferential portion 483 (and potentially also from the outer wall 701 or the aortic arch). Thus, if there is a desire to maintain all of the indentations 446a, 446c at the furthest away position from the outermost point on the second circumferential portion 483 of the outer surface of the inner shaft 402 (and potentially also from the outer wall 701 of the aortic arch 700), this can be achieved by positioning the pusher 440 on the inner shaft 402 as shown in FIG. 28 with one of the indentations 446b offset 90° from the vertical plane E-E. This positions the two indentations 446a, 446c (and the tabs 130 of the prosthetic valve 100 position therein) at the 5 o'clock and 1 o'clock positions, respectively, along the outer surface of the inner shaft 402. It is believed that the position of the pusher 440 in FIG. 28 may make it least likely for any of the tabs 130 to become stuck during deployment. However, as noted above, the position shown in FIG. 27 may also be acceptable, as may other positions of the pusher 440 relative to the inner shaft 402. In some embodiments, it may be desirable to ensure that none of the indentations 446 and tabs 130 are located at the 3 o'clock position of the inner shaft 402 so that none of the indentations 446 and tabs 130 are directly facing the outer wall 701 of the annulus of the aortic arch 700.

As seen in FIG. 26, when the deployment assembly 410 is in the aortic arch, the inner shaft 402 is in the U-shaped bend configuration described above and as such the axial wires 415, 416 are aligned on the vertical axis E-E (shown in FIG. 27, but the vertical axis E-E is going into/out of the page in FIG. 26). With reference to FIG. 27, in this position the inner shaft 402 has an outer surface 408 having a first portion 409 that faces inwardly towards itself and therefore faces the inner wall 702 of the aortic arch 700 during the valve replacement procedure and a second portion 410 that faces outwardly away from itself and that is configured to face the outer wall 701 of the aortic arch during the valve replacement procedure. As noted herein, none of the indentations 446 are aligned with or located along the second portion 410 of the outer surface 408 of the inner shaft 402. Rather, all of the indentations 446 are circumferentially offset from being in direct alignment with the second portion 410 of the outer surface 408, which also places the indentations 446 and tabs 130 in a circumferentially offset position relative to the outer wall 701 of the aortic arch 700.

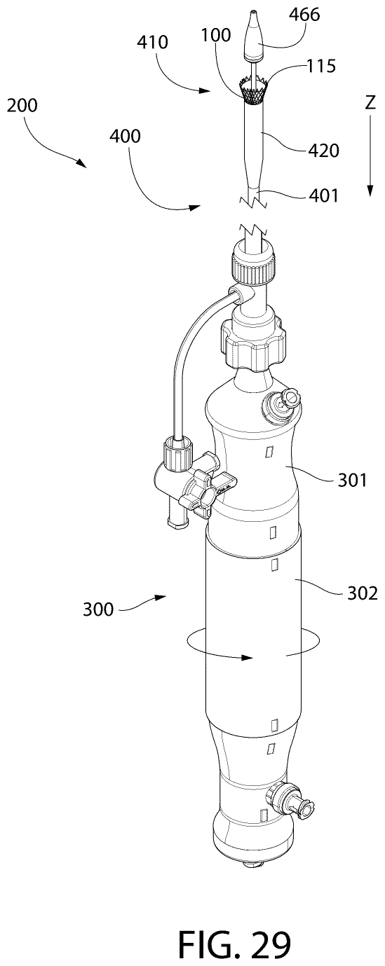

Referring to FIG. 29, once the deployment assembly 410 is properly positioned within the aorta or aortic arch of the patient, the surgeon/user can begin to deploy the prosthetic valve 100 from the catheter apparatus 100. This is accomplished by the surgeon/user rotating the rotating member 302 of the handle assembly 300 in the first rotational direction, as indicated by the arrow. As discussed in detail above, rotating the rotating member 302 of the handle assembly 300 in the first rotational direction results in the slider 303 moving in the proximal axial direction Z within the handle cavity 309, the outer shaft 401 of the catheter assembly 400 moving in the proximal axial direction Z due to its coupling to the slider 303, and the outer sheath 420 of the catheter assembly 400 moving in the proximal axial direction Z due to its coupling to the outer sheath 420.

In FIG. 29, the surgeon/user has rotated the rotating member 302 of the handle assembly 300 a particular degree or amount such that the outer sheath 420 has moved proximally a sufficient distance to expose a distal-most portion of the prosthetic valve 100 which includes the distal end 115 of the prosthetic valve 100. As the outer sheath 420 moves in the proximal axial direction Z. the nose cone 466 and the prosthetic valve 100 remain stationary. As such, this movement of the outer sheath 420 begins the process of unsheathing the prosthetic valve 100 so that it can be entirely removed from the sheath cavity 421 and implanted in the patient. In FIG. 29, the prosthetic valve 100 is not yet deployed in the patient. The surgeon could at this point potentially rotate the rotating member 302 of the handle assembly 300 in the opposite rotational direction to resheathe the exposed distal-most portion of the prosthetic valve 100.

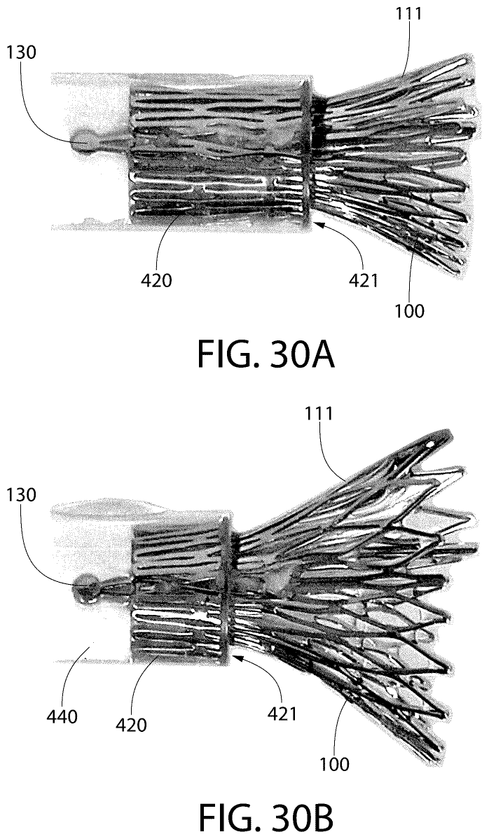

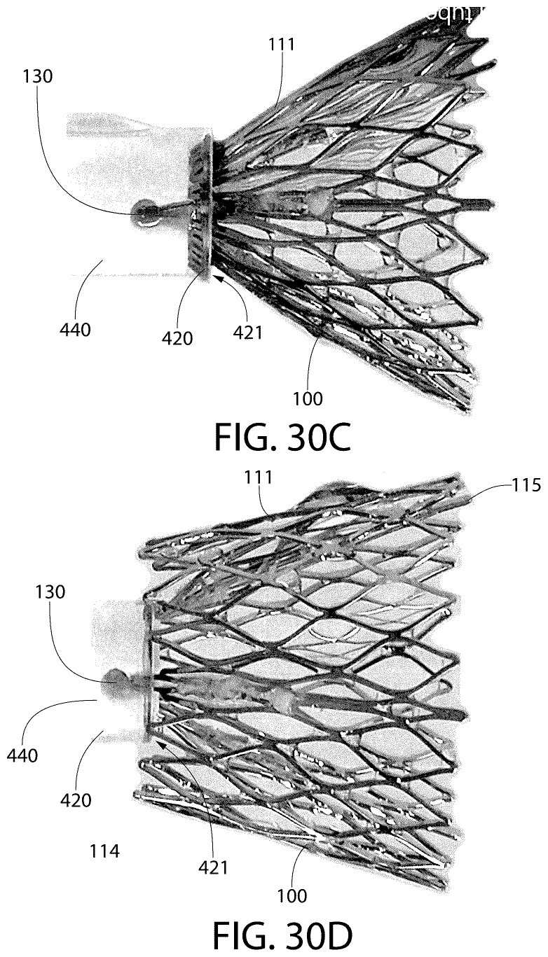

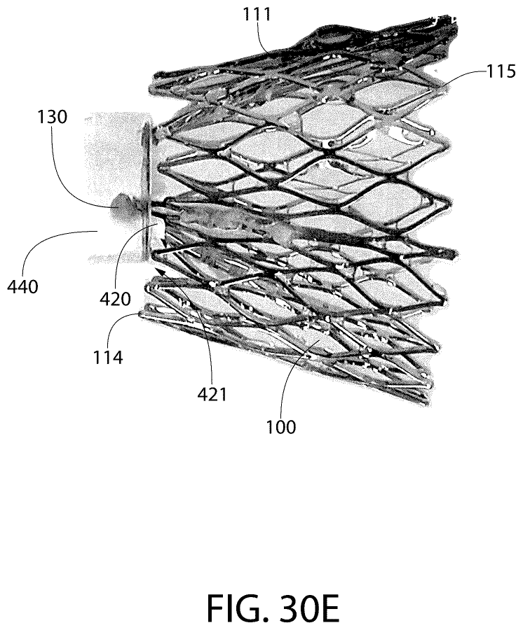

FIGS. 30A-30E are pictorial representations of the prosthetic valve 100 being deployed up to a position whereby the entirety of the tubular body portion 111 of the prosthetic valve 100 has been removed from the sheath cavity 421 of the outer sheath 420, but the tabs 130 remain located in the sheath cavity 421 of the outer sheath 420 (FIG. 30E). As the outer sheath 420 continues to be moved in the proximal axial direction Z due to the rotation of the rotating member 302 of the handle assembly 300, more of the prosthetic valve 100 becomes unsheathed from the outer sheath 420. Furthermore, as the prosthetic valve 100 becomes unsheathed from the outer sheath 420, the prosthetic valve 100 expands radially outwards in an attempt to return to its natural, non-compressed state. This is the reason that the tubular body portion 111 is angled as it extends from the outer sheath 420 because the tubular body portion 111 is automatically expanding back to its natural, expanded state when the radial compression provided by the outer sheath 420 is removed.

In FIGS. 30D and 30E, only the tabs 130 of the prosthetic valve 100 remain in the outer sheath 420 and the rest of the prosthetic valve 100, including the entirety of the tubular body portion 111, is outside of the sheath cavity 421 of the sheath 420. In this stage of the deployment procedure, despite the fact that the entire tubular body portion 111 of the prosthetic valve 100 is located outside of the sheath cavity 421, the tubular body portion 111 is not fully deployed and not fully expanded. Rather, while the distal end 115 of the tubular body portion 111 is likely in its fully expanded state, the tubular body portion 111 is conical in shape as it extends from the distal end 115 towards the proximal end 114. That is, other than at the distal end 115, or the distal-most portion of the tubular body portion 111, the prosthetic valve 100 remains at least slightly radially compressed as compared to its natural, fully expanded state when not under any compression. In this position, it is possible that the distal-most portion of the prosthetic valve 100 may contact the inner surface of the walls of the aorta or the native valve which is being replaced. However, the remainder of the prosthetic valve 100 remains spaced apart from the walls of the aorta or the native valve because the remainder of the prosthetic valve 100 has not yet expanded to its fully expanded state. Contact between the prosthetic valve 100 and the walls of the aorta is uneven until the tabs are released 130 from the sheath cavity 421.

It should be appreciated that the annulus of the aorta is not usually circular, and is often elliptical. When the annulus is elliptical, there will be greater contact between the prosthetic valve 100 and the annulus of the aorta in the short axis region than in the long axis region of the elliptical shape. However, regardless of whether the annulus is elliptical or circular, the entire tubular body portion 111 of the prosthetic valve 100 will contact the annulus once the tabs 130 are released from the sheath cavity 421. In some embodiments, when the entire tubular body portion 111 of the prosthetic valve 100 is deployed and only the tabs 130 remain in the sheath cavity 421, no more than 70% of the tubular body portion 111 of the prosthetic valve 100 is fully deployed and/or in its fully expanded state. In some embodiments, when the entire tubular body portion 111 of the prosthetic valve 100 is deployed and only the tabs 130 remain in the sheath cavity 421, no more than 50% of the tubular body portion 111 of the prosthetic valve 100 is fully deployed and/or in its fully expanded state. By this, it is meant that no more than 70% or no more than 50% of the length of the tubular body portion 111 of the prosthetic valve 100 (with the length measured between the proximal 114 and distal ends 115) is in contact with the annulus of the aorta. By “fully expanded state” it is meant that the tubular body portion 111 has returned from its radially compressed state to its natural state where it is not under any radial compression. Furthermore, the percentages noted refers to the length of the body portion 111 that has returned to its natural, non-compressed state. That is, if no more than 70% of the body portion 111 is in the fully expanded state, then no more than 70% of the length of the body portion 111 measured between the proximal end distal ends 114, 115 has returned to its non-compressed state (such that it is not compressed radially at all).

In some embodiments, when the annulus of the aortic arch is more circular, when only the tabs remain in the sheath cavity 421 as shown in FIGS. 30D and 30E, approximately between 30% and 60%, and more specifically between 40% and 50%, and still more specifically approximately 45% of the length of the tubular body 111 of the prosthetic valve 100 is in contact with the annulus of the aortic arch. In some embodiments, where the annulus is more elliptical, when only the tabs remain in the sheath cavity 421 as shown in FIGS. 30D and 30E, approximately between 60% and 80%, and more specifically between 65% and 75%, and still more specifically between 68% and 70%, and more specifically approximately 69% of the length of the tubular body 111 of the prosthetic valve 100 is in contact with the annulus of the aortic arch. However, in the elliptical situation, because of the non-circular shape of the annulus, the contact may not be around the full circumference of the prosthetic valve 100 (the contact may be uneven).

Thus, it should be appreciated that regardless of the shape (circular or elliptical) of the annulus, when the full length of the tubular body 111 is removed from the sheath cavity 421 of the outer sheath 420 and only the tabs 130 of the prosthetic valve 100 remain located in the sheath cavity 421 of the outer sheath 420, no more than 70% of the prosthetic valve 100 is fully deployed and in a fully expanded state. This means that no more than 70% of the length of the prosthetic valve 100 is in contact with the annulus of the aortic arch when everything except the tabs 130 have been released from the sheath cavity 421 of the outer sheath 420.

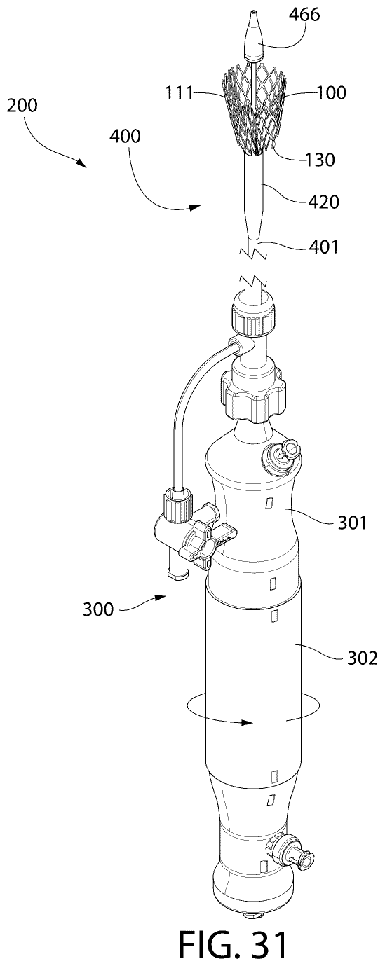

In FIG. 31, the surgeon/user has continued the deployment of the prosthetic valve 100 beyond the position shown in FIG. 30E by continuing to rotate the rotating member 302 of the handle assembly 300 in the first rotational direction. As such, the outer sheath 420 continues to move in the proximal axial direction Z and more of the prosthetic valve 100 emerges from the sheath cavity 421. In FIG. 31, the outer sheath 420 has moved in the proximal axial direction Z a sufficient distance such that one of the tabs 130 of the prosthetic valve 100 has been release from the outer sheath 420 of the catheter assembly 400. Even in this position, it should be noted that the prosthetic valve 100 is still not fully deployed and is still not in its fully expanded state as two of the tabs 130 remain in the outer sheath 420.

It should be appreciated that despite the fact that the tabs 130 all have an identical length, the tabs 130 are removed from the outer sheath 420 sequentially, one at a time. In no case is more than one of the tabs 130 removed from the outer sheath 420 at a time, such that there is no simultaneous removal of multiple ones of the tabs 130 of the prosthetic valve 100 from the outer sheath 420. Without intending to be bound by theory, it is believed that the sequential release of the tabs 130 from the sheath cavity 421 during deployment of the prosthetic valve 100 may be due, at least in part, to the orientation of the pusher 440 relative to the inner shaft 402. Regardless of the reason, the fact remains that the tabs 130 may be released from the pusher 440 and sheath cavity 421 sequentially, one at a time, and never simultaneously in accordance with some of the embodiments described herein. This provides the surgeon with a lot of control during the deployment and installation of the prosthetic valve 100.

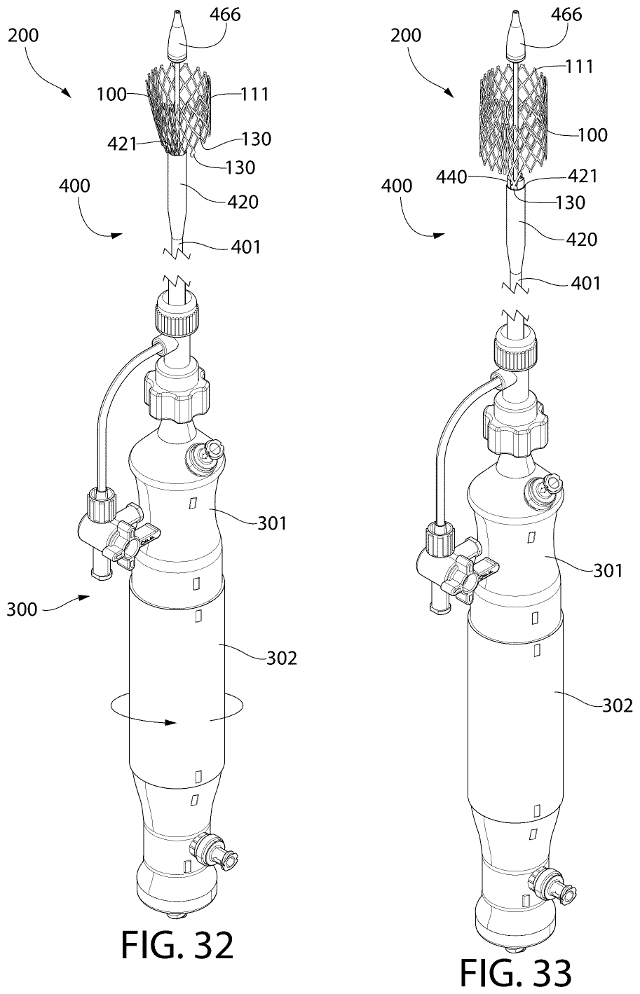

Referring to FIG. 32, the surgeon/user continues to rotate the rotating member 302 of the handle assembly 300 until the second tab 130 of the prosthetic valve 100 is removed from the sheath cavity 421 of the outer sheath 420. As such, more of the prosthetic valve 100 expands into its fully expanded state. In some embodiments, upon the second tab 130 being released from the pusher 440 and the sheath cavity 421, the prosthetic valve 100 may be in its fully expanded state. In other embodiments, it may not be until all three of the tabs 130 have been released that the prosthetic valve 100 is in its fully expanded state. In that regard, FIG. 33 illustrates the stage in the deployment process whereby all three of the tabs 130 have been release from the sheath cavity 421 and separated/detached from the pusher 440. At this point, the prosthetic valve 100 is fully deployed and in its fully expanded state along its entire length and is completely implanted in the user's body (along the aortic valve region).

It is worth repeating that during the deployment and installation of the prosthetic valve 100 into the patient, the tabs 130 of the prosthetic valve 100 are released from the sheath cavity 421 and the pusher 440 sequentially rather than simultaneously despite the fact that the tabs 130 are all the same length as described herein. Thus, the tabs 130 are released one at a time until all of the tabs 130 have been released. This feature of the inventive catheter system (which includes the catheter apparatus 200 and the prosthetic valve 100) allows the prosthetic valve 100 to be repositioned, recaptured, and/or retrieved during the implantation/installation procedure. In particular, this feature allows the surgeon to maintain control of the prosthetic valve 100 and its deployment until the very last tab 130 is released from the pusher 440 and sheath cavity 421. Thus, even after the first tab 130 has been released from the pusher 440, and perhaps after the second tab 130 has been released from the pusher 440, the catheter apparatus 100 may be capable of repositioning, recapturing, and/or retrieving the prosthetic valve 100 as needed. This allows the surgeon or other user to have optimal control for proper positioning of the prosthetic valve 100 during a procedure.

In some embodiments, the systems and methods described herein involve a self-expanding prosthetic valve that comprises: a frame comprising a tubular body; a valve component disposed within and anchored to the tubular body of the frame, the valve component comprising: an annular sleeve having an annular inner wall that forms a fluid passageway along an axis from an inlet edge to an outlet edge, an annular cuff that is concentric to and surrounds the annular inner wall and extends from the inlet edge toward the outlet edge; and an annular belt positioned between the annular inner wall and the annular cuff; wherein the annular belt is concentric to and circumferentially surrounds the annular inner wall; and wherein the annular sleeve and the annular belt are formed of a material selected from the group consisting of a biological tissue and a biocompatible polymer.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A catheter apparatus comprising:
a handle assembly comprising:
a handle housing comprising a longitudinal axis, an outer surface and an inner surface that defines a handle cavity;
a rotating member positioned around a portion of the outer surface of the handle housing and configured to rotate relative to the handle housing, the rotating member comprising a threaded inner surface;
a slider at least partially positioned within the handle cavity and comprising a threaded portion that mates with the threaded inner surface of the rotating member;
an inlet port defining a passageway into the handle cavity; and
a flushing tube having a coiled configuration, the flushing tube comprising a first end fluidly coupled to the inlet port, a second end fluidly coupled to the slider, and a lumen extending from the first end to the second end; and
wherein rotation of the rotating member causes the slider to move axially within the handle cavity, and wherein the flushing tube compresses as the slider moves in a proximal axial direction and expands as the slider moves in a distal axial direction.

2. The catheter apparatus according to claim 1 wherein an axial length of the flushing tube increases and decrease as the slider moves axially within the handle cavity due to the coupling of the flushing tube to the slider.

3. The catheter apparatus according to claim 1 wherein a pitch of the flushing tube increases as the slider moves in the distal axial direction and decreases as the slider moves in the proximal axial direction.

4. The catheter apparatus according to claim 1 wherein the slider comprises a top surface, a bottom surface, a through-hole extending from the top surface to the bottom surface, and a flushing passageway extending from an inlet in the bottom surface to an outlet that is in fluid communication with the through-hole, wherein the first end of the flushing tube nests within the inlet port of the handle assembly and the second end of the flushing tube nests within the flushing passageway of the slider so that a fluid introduced into the inlet port of the handle assembly flows through the flushing tube and into the flushing passageway.

5. (canceled)

6. The catheter apparatus according to claim 4 further comprising a catheter assembly operably coupled to the handle assembly, the catheter assembly comprising an outer shaft and an inner shaft located at least partially within an interior of the outer shaft, wherein the inner shaft extends entirely through the through-hole in the slider and the outer shaft comprises a proximal end that is located within the through-hole in the slider; wherein the proximal end of the outer shaft is positioned adjacent and distal to the outlet of the flushing passageway, and wherein the outlet of the flushing passageway is positioned radially inward of the outer shaft of the catheter assembly so that fluid introduced into the inlet port of the handle assembly flows between the inner and outer shafts to remove any air located between the inner and outer shafts.

7. (canceled)

8. The catheter apparatus according to claim 6 wherein the catheter assembly comprises an outer sheath defining a sheath cavity, a distal tip assembly that closes a distal end of the outer sheath, and a pusher located within the sheath cavity, the outer shaft extends from the proximal end to a distal end, the proximal end being coupled to the slider and the distal end being coupled to the outer sheath so that as the slider moves axially within the handle cavity in response to rotation of the rotating member, the outer shaft and the outer sheath also move axially, wherein rotation of the rotating member in a first rotational direction causes the slider, the outer shaft, and the outer sheath to move in the proximal axial direction to expose the pusher and deploy a prosthetic aortic valve that is configured to be positioned within the sheath cavity between the pusher and the distal tip assembly.

9.-11. (canceled)

12. The catheter apparatus according to claim 1 wherein the slider comprises a hub portion located within the handle cavity and at least one engagement portion that terminates with the threaded portion of the slider, the engagement portion extending from the hub portion and being at least partially located outside of the handle cavity for engagement with the threaded inner surface of the rotating member.

13. (canceled)

14. A catheter apparatus comprising:
a catheter assembly comprising:
an inner shaft comprising a lumen;
a first axial wire and a second axial wire disposed within the lumen of the inner shaft, wherein when the inner shaft is bent into a U-shaped bend configuration the first and second axial wires are aligned on a vertical plane; and
a pusher comprising a plurality of indentations that are configured to receive tabs of a frame of a prosthetic aortic valve, wherein the pusher is coupled to the inner shaft so that one of the plurality of indentations is angularly offset between 80° and 100° from the vertical plane.

15. The catheter apparatus according to claim 14 wherein the inner shaft comprises a proximal end and a distal end, and wherein the pusher is coupled to the shaft at a position between the proximal and distal ends and closer to the distal end than the proximal end.

16. The catheter apparatus according to claim 14 wherein the one of the plurality of indentations is angularly offset 90° from the vertical plane.

17. The catheter apparatus according to claim 14 further comprising:
a handle assembly comprising:
a handle housing comprising a proximal portion comprising an inner surface having a threaded portion and a first angular alignment feature;
a proximal cap comprising a threaded portion that mates with the threaded portion of the inner surface of the proximal portion of the handle housing to couple the proximal cap to the handle housing; and
a proximal key coupled to the proximal cap, the proximal key comprising a second angular alignment feature that mates with the first angular alignment feature to maintain the proximal key at a set angular orientation relative to the handle housing; and
wherein a proximal end of the inner shaft of the catheter assembly is fixedly coupled to the proximal key so that the set angular orientation of the proximal key relative to the handle housing dictates the angular orientation of the inner shaft and the pusher of the catheter assembly relative to the handle housing.

18. (canceled)

19. The catheter apparatus according to claim 14 further comprising a handle assembly comprising:
a handle housing comprising an outer surface and an inner surface that defines a handle cavity;
a rotating member positioned around a portion of the outer surface of the handle housing and configured to rotate relative to the handle housing, the rotating member comprising a threaded inner surface that is spaced apart from the outer surface of the handle housing; and
a slider at least partially positioned within the handle cavity, the slider comprising a threaded portion that mates with the threaded inner surface of the rotating member so that rotation of the rotating member in a first rotational direction causes the slider to move in a distal axial direction and rotation of the rotating member in a second rotational direction causes the slider to move in a proximal axial direction.

20. (canceled)

21. The catheter apparatus according to claim 14 wherein the pusher comprises a proximal end and a distal end, each of the indentations extending from the distal end of the pusher to a proximal end surface, and wherein the proximal end surface is sloped downwardly in a direction towards the proximal end of the pusher to facilitate disengagement of the tabs of the frame of the prosthetic aortic valve from the pusher during deployment.

22. The catheter apparatus according to claim 14 wherein the inner shaft in the U-shaped bend configuration comprises an outer surface having a first portion that faces inwardly towards itself and that is configured to face an inner wall of an aorta during a valve replacement procedure and a second portion that faces outwardly away from itself and that is configured to face an outer wall of the aorta during a valve replacement procedure, and wherein none of the plurality of indentations are located along the second portion of the outer surface of the inner shaft.

23. The catheter apparatus according to claim 14 further comprising an axis perpendicular to the vertical plane that intersects one of the plurality of indentations of the pusher, wherein the axis perpendicular to the vertical plane intersects the one of the plurality of indentations of the pusher and does not intersect any of the other indentations of the pusher.

24. (canceled)

25. (canceled)

26. A catheter apparatus comprising:
a handle assembly; and
a catheter assembly coupled to the handle assembly, the catheter assembly comprising a pusher configured for engagement with a prosthetic aortic valve to facilitate implantation of the prosthetic aortic valve into a patient, the pusher comprising at least one indentation that is configured to receive a tab of a frame of the prosthetic aortic valve, the at least one indentation comprising a proximal end surface that is sloped.

27. The catheter apparatus according to claim 26 wherein the pusher comprises a distal end, a proximal end, and a longitudinal axis extending between the distal and proximal ends, the at least one indentation being elongated axially from the distal end of the pusher to the proximal end surface, and wherein the proximal end surface is sloped downwardly in a direction away from the distal end and towards the proximal end of the pusher.

28. The catheter apparatus according to claim 26 wherein the at least one indentation comprises a plurality of the indentations that are circumferentially spaced apart from one another.

29. The catheter apparatus according to claim 26 wherein the pusher comprises a distal end, a proximal end, and an outer surface, and wherein the at least one indentation forms a recess in the outer surface that terminates in a floor that is recessed relative to the outer surface, and wherein the proximal end surface of the at least one indentation is oriented at an oblique angle relative to the floor of the recess.

30. The catheter apparatus according to claim 29 wherein the at least one indentation comprises an entry section adjacent to the distal end of the pusher, a nesting section adjacent to the proximal end surface of the at least one indentation, and a neck section located between the entry and nesting sections, the neck section having a reduced width relative to the entry and nesting sections, wherein the at least one indentation comprises a longitudinal axis and a sidewall surface that extends from the floor of the at least one indentation to the outer surface of the pusher, the sidewall surface comprising a first portion located on a first side of the longitudinal axis and a second portion located on a second side of the longitudinal axis, and further comprising a first tab protruding from the first portion of the sidewall surface and a second tab protruding from the second portion of the sidewall surface to define the neck section of the at least one indentation, and wherein each of the first and second tabs comprises a distal surface that faces the distal end of the pusher, the distal surfaces of the first and second tabs being sloped downwardly in a direction away from the distal end of the pusher.

31.-106. (canceled)
